(19) United States
(12) Patent Application Publication
(10) Pub. No.: US 2012/0065571 A1
(43) Pub. Date: Mar. 15, 2012

(54) EXPANDABLE PYLORIC ANCHORS AND METHODS FOR SECURING INTESTINAL BYPASS SLEEVES

(75) Inventors: Paul J. Thompson, Minnetonka, MN (US); Kedar R. Belhe, Minnetonka, MN (US); Alex Grafov, Eden Prairie, MN (US)

(73) Assignee: MetaModix, Inc., Plymouth, MN (US)

(21) Appl. No.: 13/298,867

(22) Filed: Nov. 17, 2011

Related U.S. Application Data
(63) Continuation-in-part of application No. 12/752,697, filed on Apr. 1, 2010.
Application No. 12/752,697 is a continuation-in-part of application No. 12/833,605, filed on Jul. 9, 2010.
Application No. 12/833,605 is a continuation-in-part of application No. 12/986,268, filed on Jan. 7, 2011.
(60) Provisional application No. 61/458,060, filed on Nov. 17, 2010.
(60) Provisional application No. 61/211,853, filed on Apr. 3, 2009.
(60) Provisional application No. 61/270,588, filed on Jul. 10, 2009.
(60) Provisional application No. 61/335,472, filed on Jan. 7, 2010.

(51) Int. Cl. A61M 5/00

(52) U.S. Cl. 604/8

(57) ABSTRACT

A gastrointestinal device for implanting within a patient's gastrointestinal tract includes first and second expandable anchoring rings configured to be disposed on either side of a patient's pylorus. The first expandable anchoring ring has a collapsed configuration and an expanded configuration, wherein in the expanded configuration the first expandable anchoring ring has a diameter larger than a maximum opening diameter of the pylorus. The second expandable anchoring ring also has a collapsed configuration and an expanded configuration, wherein in the expanded configuration the second expandable anchoring ring has a diameter larger than a maximum opening diameter of the pylorus. An intestinal bypass sleeve couples the first expandable anchoring ring and the second expandable anchoring ring, so as to allow a distance between the anchoring rings to exceed a width of the pylorus.

### CROSS-REFERENCE TO RELATED APPLICATION

This application claims the benefit under 35 U.S.C. section 119(e) to U.S. provisional patent application 61/458,060, filed Nov. 17, 2010, which is herein incorporated by reference in its entirety. This application is also a continuation-in-part of each of the following applications, the disclosures of which are each hereby incorporated by reference: (1) U.S. patent application Ser. No. 12/752,697, filed Apr. 1, 2010, entitled “Modular Gastrointestinal Prostheses,” which claims the benefit of U.S. provisional patent application 61/211,853, filed Apr. 3, 2009; (2) U.S. patent application Ser. No. 12/833,605, filed Jul. 9, 2010, entitled “External Anchoring Configuration for Modular Gastrointestinal Prostheses,” which claims the benefit of U.S. provisional patent application 61/270,588, filed Jul. 10, 2009; and (3) U.S. patent application Ser. No. 12/986,268, filed Jan. 7, 2011, entitled “Gastrointestinal Prostheses Having Partial Bypass Configurations,” which claims the benefit of U.S. provisional patent application 61/335,472, filed Jan. 7, 2010.

### TECHNICAL FIELD

The present invention relates to implants placed within gastrointestinal systems, including the esophagus, the stomach and the intestines. In particular it relates to implant systems having components implantable and removable using endoscopic techniques for treatment of obesity, diabetes, reflux, gastroparesis and other gastrointestinal conditions.

### BACKGROUND

Bariatric surgery procedures, such as sleeve gastrectomy, the Rouen-Y gastric bypass (RYGB) and the bileo-pancreatic diversion (BPD), modify food intake and/or absorption within the gastrointestinal system to effect weight loss in obese patients. These procedures affect metabolic processes within the gastrointestinal system, by either short circuiting certain natural pathways or creating different interaction between the consumed food, the digestive tract, its secretions and the neuro-hormonal system regulating food intake and metabolism. In the last few years there has been a growing clinical consensus that obese patients who undergo bariatric surgery see a remarkable resolution of their type-2 Diabetes Mellitus (T2DM) soon after the procedure. The remarkable resolution of diabetes after RYGB and BPD typically occurs too fast to be accounted for by weight loss alone, suggesting there may be a direct impact on glucose homeostasis. The mechanism of this resolution of T2DM is not well understood, and it is quite likely that multiple mechanisms are involved.

One of the drawbacks of bariatric surgical procedures is that they require fairly invasive surgery with potentially serious complications and long patient recovery periods. In recent years, there is an increasing amount of ongoing effort to develop minimally invasive procedures to mimic the effects of bariatric surgery using minimally invasive procedures. One such procedure involves the use of gastrointestinal implants that modify transport and absorption of food and organ secretions. For example, U.S. Pat. No. 7,476,256 describes an implant having a tubular sleeve with anchoring barbs, which offer the physician limited flexibility and are not readily removable or replaceable. Moreover, stents with active fixation means, such as barbs that penetrate into surrounding tissue, may potentially cause tissue necrosis and erosion of the implants through the tissue, which can lead to complications, such as systemic infection. Also, due to the intermittent peristaltic motion within the digestive tract, implants such as stents have a tendency to migrate.

Gastroparesis is a chronic, symptomatic disorder of the stomach that is characterized by delayed gastric emptying in the absence of mechanical obstruction. The cause of gastroparesis is unknown, but it may be caused by a disruption of nerve signals to the intestine. The three most common etiologies are diabetes mellitus, idiopathic, and postsurgical. Other causes include medication, Parkinson's disease, collagen vascular disorders, thyroid dysfunction, liver disease, chronic renal insufficiency, and intestinal pseudo-obstruction. The prevalence of diabetic gastroparesis (DGP) appears to be higher in women than in men, for unknown reasons.

Diabetic gastroparesis affects about 40% of patients with type 1 diabetes and up to 30% of patients with type 2 diabetes and especially impacts those with long-standing disease. Both symptomatic and asymptomatic DGP seem to be associated with poor glycemic control by causing a mismatch between the action of insulin (or an oral hypo-glycemic drug) and the absorption of nutrients. Treatment of gastroparesis depends on the severity of the symptoms.

U.S. Pat. No. 5,820,584 to Crabb discloses use of two rings, one on each side of the pylorus to anchor an intestinal bypass sleeve in the duodenum. The rings disclosed in Crabb were disclosed to be a collapsible yet resilient material, yet no specific material is disclosed for the rings.

### SUMMARY

According to various embodiments, the present invention is a method and apparatus to place and anchor an intestinal bypass sleeve with the pylorus, duodenum and/or jejunum. The gastrointestinal implant can be inserted endoscopically in combination with a delivery catheter. A device that permanently holds open the pylorus may provide help in allowing the stomach to empty more rapidly in patients with gastroparesis. According to various embodiments, the present invention, with a short bypass sleeve or no bypass sleeve, can be used to hold open the pylorus and may help to reduce the symptoms of gastroparesis, by allowing the stomach contents to exit the stomach easier through the pylorus into the duodenum.

Nitinol material can undergo an elastic recovery from a maximum strain of up to 8.5% strain before non-recoverable plastic deformation occurs. Nitinol rings made from wire in the diameter range of 0.020 to 0.027 inch results in a large constrained diameter of the sleeves and anchors on a catheter in the range of 10 to 15 mm, which is undesirable. This large diameter Nitinol wires may also result in fatigue properties and a fatigue life inferior to that obtained using a wire of a smaller diameter in construction of the anchors. According to various embodiments, the present invention is a ring and other anchor configurations constructed of stranded or highly stranded Nitinol wire. The wire diameter of the exemplary embodiment may be in the range of 0.0005 inch up to 0.0030 inch. In other embodiments, the Nitinol wire is made of strands having an outer diameter of up to 0.015 inch. In various embodiments, the gastrointestinal implant includes a flexible sleeve and an expandable anchor attached to the proximal end of the sleeve.

The present invention, according to some embodiments, is a gastrointestinal device for implanting within a pylorus, a duodenal bulb, and a duodenum of a patient's gastrointestinal tract. The implant includes a first expandable anchoring ring formed from stranded wire, the first expandable anchoring ring having a collapsed configuration and an expanded configuration, wherein in the expanded configuration the first expandable anchoring ring has a diameter larger than a maximum opening diameter of the pylorus. It further includes a second expandable anchoring ring formed from stranded wire, the second expandable anchoring ring having a collapsed configuration and an expanded configuration, wherein in the expanded configuration the second expandable anchoring ring has a diameter larger than a maximum opening diameter of the pylorus. An intestinal bypass sleeve is coupled to the first expandable anchoring ring and the second expandable anchoring ring, so as to allow a distance between the anchoring rings to exceed a width of the pylorus.

The present invention, according to some embodiments, is an anchoring device for implanting within a pylorus for treating gastroparesis. The device includes a first expandable anchoring ring having a collapsed configuration and an expanded configuration, wherein in the expanded configuration the first expandable anchoring ring has a diameter larger than a maximum opening diameter of the pylorus, and a second expandable anchoring ring having a collapsed configuration and an expanded configuration, wherein in the expanded configuration the second expandable anchoring ring has a diameter larger than a maximum opening diameter of the pylorus. The two anchoring rings are connected by a pyloric portion, the pyloric portion having a length greater than a width of the pylorus, such that the first expandable anchoring ring may be disposed on a first side of the pylorus and a second expandable anchoring ring may be disposed on a second side of the pylorus, and the pyloric portion is configured to resist closure of the pylorus.

The present invention, according to some embodiments, is a method for treating gastroparesis comprising compressing a first expandable anchoring ring in a delivery tool, the first expandable anchoring ring having a collapsed configuration and an expanded configuration, wherein in the expanded configuration the first expandable anchoring ring has a diameter larger than a maximum opening diameter of the pylorus, compressing a second expandable anchoring ring in the delivery tool, the second expandable anchoring ring having a collapsed configuration and an expanded configuration, wherein in the expanded configuration the second expandable anchoring ring has a diameter larger than a maximum opening diameter of the pylorus, advancing the delivery tool to a pylorus of a patient, deploying the second expandable anchoring ring on a first side of the pylorus, deploying the first expandable anchoring ring on a second side of the pylorus, and coupling a valve coupled to a pyloric portion extending between the first and second expandable anchoring rings, the valve adapted to allow flow through the pyloric portion in a first direction and to resist flow through the pyloric portion in a second direction.

While multiple embodiments are disclosed, still other embodiments of the present invention will become apparent to those skilled in the art from the following detailed description, which shows and describes illustrative embodiments of the invention. Accordingly, the views and detailed description are to be regarded as illustrative in nature and not restrictive.

### BRIEF DESCRIPTION OF THE DRAWINGS

FIG. 1 is a sectional view of a portion of the digestive tract in the body. An intestinal bypass sleeve is implanted in the duodenum from the pylorus to the ligament of treitz or into the jejunum. The sleeve is held in place at the pylorus by two expandable anchors one each side of the pylorus.

FIGS. 2-4 are sectional views showing various embodiments of an expandable anchor as used to anchor the intestinal bypass sleeve at the pylorus.

FIG. 5A shows an alternative embodiment of expandable anchor that can be used to anchor the intestinal bypass sleeve at the pylorus. The anchor has an integral loop that can be grabbed to recover the device to remove it.

FIG. 5B shows an alternative embodiment of expandable anchor that can be used to anchor the intestinal bypass sleeve at the pylorus. The anchor has two integral loops that can be grabbed to recover the device to remove it.

FIG. 6 shows a sleeve implanted into a pylorus and duodenal bulb and duodenum. The rings have an outer diameter selected such that there is minimal or no contact force between the rings and the stomach pyloric antrum and the duodenal bulb.

FIG. 7 is a sectional view of an embodiment of the invention implanted into a pylorus and duodenal bulb and duodenum. A ring-like structure is disposed on the stomach side of the pylorus and a conventional stent is disposed on the duodenal side of the pylorus.

FIG. 8 is a sectional view of an embodiment of the invention implanted into a pylorus and duodenal bulb and duodenum. A ring-like structure is disposed on the stomach side of the pylorus and a ring-like structure is disposed on the duodenal side of the pylorus. Two smaller diameter ring-like structures are disposed within the area of the pylorus opening.

FIG. 9 is a sectional view of an embodiment of the invention implanted into a pylorus and duodenal bulb and duodenum. The rings are sized sufficiently large in diameter to engage the wall of the stomach pyloric antrum and the duodenal bulb.

FIG. 10 is a sectional view of an embodiment of the invention implanted into a pylorus and duodenal bulb and duodenum. The rings are sized sufficiently large in diameter to engage the wall of the stomach pyloric antrum and the duodenal bulb. The central portion of the device is constructed of a rigid, fixed-diameter cylinder.

FIG. 11 is a sectional view of an embodiment of the invention implanted into a pylorus and duodenal bulb and duodenum. The rings are sized sufficiently large in diameter to engage the wall of the stomach pyloric antrum and the duodenal bulb. The central portion of the device is constructed of an expandable diameter cylinder having a stent-like configuration or construction. The cylinder is constructed to have sufficient radial strength to resist compression by the pylorus when the pylorus attempts to close.

FIG. 12 is a sectional view of an embodiment of the invention implanted into a pylorus and duodenal bulb and duodenum. The rings are sized sufficiently large in diameter to engage the wall of the stomach pyloric antrum and the duodenal bulb. The central portion of the device is constructed of a rigid, fixed-diameter cylinder. The central lumen of device has a one way anti-reflux valve. The anti-reflux valve allows for unobstructed flow in the direction of the stomach antrum to the pylorus, but limits flow in the reverse direction. The anti-reflux valve can be constructed of a duck bill design with two flexible leaflets, or may utilize other designs such as a tri leaflet or quad leaflet.

FIG. 13 is a sectional view of an embodiment of the invention implanted into a pylorus and duodenal bulb and duodenum. The rings are sized sufficiently large in diameter to engage the wall of the stomach pyloric antrum and the duodenal bulb. The central portion of the device is constructed of a rigid, fixed-diameter cylinder. The central lumen of device has a one way anti-reflux valve. The anti-reflux valve allows for unobstructed flow in the direction of the stomach antrum to the pylorus, but limits flow in the reverse direction. The anti-reflux valve can be constructed of a ball and cage design, or may utilize other constructions such as a rigid bi-leaflet valve.

FIG. 14 is a sectional view of an embodiment of the invention implanted into a pylorus and duodenal bulb and duodenum. Two ring-like structures are on the stomach side of the pylorus and two ring-like structures are implanted on the duodenal side of the pylorus.

FIG. 15 is a sectional view of an embodiment of the invention implanted into a pylorus and duodenal bulb and duodenum. The rings are sized sufficiently small that there is minimal or no contact force between the rings and the stomach pyloric antrum and the duodenal bulb. A removal loop for the anchor is attached to the ring on the stomach side of the pylorus. The removal loop is a small diameter wire loop which can be grabbed easily by a forceps or snare and allows the device to be collapsed and pulled into a recovery catheter for removal from the human body.

FIG. 16 is a sectional view of an embodiment of the invention implanted into a pylorus and duodenal bulb and duodenum. A ring-like structure is on the stomach side of the pylorus and a conventional stent is on the duodenal side of the pylorus. A removal loop for the anchor is attached to the ring on the stomach side of the pylorus.

FIG. 17 shows an implant including two rings constructed of stranded Nitinol. The two rings are coupled by longitudinal links. The longitudinal links may be constructed of solid Nitinol wire or stranded Nitinol wire or stranded other material such as stainless steel, Elgiloy, MP35N or L605. The longitudinal links may extend parallel to the longitudinal axis of the device or may run at an angle to the longitudinal axis. The longitudinal links may be constructed integral with the anchor rings, from the same wire and wound at the same time or they may be made discretely and simply attached to the anchor rings. A removal loop is attached to one of the anchors (rings).

FIG. 18 shows an implant including two rings constructed of stranded Nitinol. The two rings are coupled by longitudinal links; the longitudinal links are curved to form a central portion which has a smaller diameter the anchor rings. The longitudinal links may be constructed of solid Nitinol wire or stranded Nitinol wire or stranded other material such as stainless steel, Elgiloy, MP35N or L605. The longitudinal links may extend parallel to the longitudinal axis of the device or may run at an angle to the longitudinal axis. The longitudinal links are curved so that the central portion of the linked structure forms a narrow diameter (waist) in the middle between the two rings. The waist may be aligned with and straddle the opening of the pylorus. The longitudinal links may be constructed integral with the anchor rings, from the same wire and wound at the same time or they may be made discretely and simply attached to the anchor rings. A removal loop is attached to one of the anchors (rings).

FIG. 19 is a sectional view of the anchor disclosed in FIG. 18 with a sleeve attached to the anchor from the proximal end to distal end. The anchor and sleeve are implanted across the pylorus from the stomach pyloric antrum to the duodenal bulb.

FIG. 20 shows an implant including two rings constructed of stranded Nitinol. The two rings are linked by longitudinal links. The longitudinal links may be constructed of solid Nitinol wire or stranded Nitinol wire or stranded other material such as stainless steel, Elgiloy, MP35N or L605. The longitudinal links may extend parallel to the longitudinal axis of the device or may run at an angle to the longitudinal axis (two set of longitudinal links may cross to form a diamond-shaped lattice work. The longitudinal links may be constructed integral with the anchor rings, from the same wire and wound at the same time or they may be made discretely and simply attached to the anchor rings. A removal loop is attached to one of the anchors (rings).

FIG. 21 is a view of an embodiment of the invention including two rings constructed of stranded Nitinol. The anchors rings may be made of two different diameters to provide for anchoring at different lumen diameters. The anchor rings are linked by longitudinal links. The longitudinal links may be constructed of solid Nitinol wire or stranded Nitinol wire or stranded other material such as stainless steel, Elgiloy, MP35N or L605. The longitudinal links may extend parallel to the longitudinal axis of the device or may run at an angle to the longitudinal axis. The longitudinal links may be constructed integral with the anchor rings, from the same wire and wound at the same time or they may be made discretely and simply attached to the anchor rings. A removal loop is attached to one of the anchors (rings).

FIG. 22 shows schematic views of an anchor ring in the open state and in the collapsed state ready to be loaded onto a delivery catheter.

FIG. 23 shows an anchor ring in the open state and in alternative collapsed states ready to be loaded onto a delivery catheter.

FIG. 24 is a sectional view of a delivery catheter.

FIG. 25 is a sectional view of multiple anchors linked by a sleeve.

FIG. 26 is a sectional view of multiple anchors linked by a sleeve. The two layers of anchors are angled at opposite directions from the longitudinal axis to form a diamond-shaped lattice.

FIG. 27 shows two anchor rings linked by a flexible membrane. The two anchor rings are also linked by a small diameter wire, near the removal loop. The small diameter wire serves to provide a rigid link between the two rings that allows easier collapse and removal of the rings.

FIG. 28 shows a removal catheter/sheath with a removal snare attached to the removal loop on the proximal anchor ring.

FIG. 29 is a schematic sectional view of two anchor rings collapsed and partially retrieved into the recovery catheter.

FIG. 30 is a sectional view of a portion of the digestive tract in the body. An intestinal bypass sleeve is implanted in the duodenum from the pylorus to the ligament of treitz or into the jejunum. The sleeve is held in place at the pylorus by two expandable anchors on each side of the pylorus. A third expandable anchor is attached to the end of the sleeve near the ligament of treitz.

FIG. 31 is a sectional view of a portion of the digestive tract in the body. An intestinal bypass sleeve is implanted in the duodenum from the pylorus to the ligament of treitz or into the jejunum. The sleeve is held in place at the pylorus by two expandable anchors on each side of the pylorus. A third expandable anchor is attached to the end of the sleeve near the ligament of treitz. A four, fifth and sixth expandable anchor are spaced between the pylorus and the ligament of treitz.

FIGS. 32A-32C show covered stents made from stranded wire having anchor rings located, respectively, on the inside of a polymer sleeve, the outside of a polymer sleeve, or within the wall thickness of the polymer sleeve.

FIG. 33 shows a covered stent as in any of FIGS. 32A-32C implanted in the human body in the trachea.

FIG. 34 shows a covered stent as in any of FIGS. 32A-32C implanted in the human body in an aortic aneurism. The stranded wire is also used as an anchor for a stent for a percutaneously or transapically placed aortic or mitral heart valve, or for a mitral valve annuloplasty ring.

FIG. 35 is a sectional view of the invention having two anchor rings attached to the device. A rigid cylinder is located in between the two anchor rings. An intestinal bypass sleeve is attached to anchors.

The cylinder length can be adjusted to change the spacing in between the two anchor rings. An anti-reflux valve is located within the rigid cylinder. The anti-reflux valve allows for free flow of the stomach contents in to the duodenum, but can reduce the back flow of duodenal contents into the stomach from the duodenum.

FIG. 36 is a sectional view of a portion of the digestive tract in the body. An intestinal bypass sleeve is implanted in the duodenum from the pylorus to the ligament of treitz. The sleeve is held in place at the pylorus by two expandable anchors one each side of the pylorus. An anti-reflux device for GERD is implanted at the gastro-esophageal (GE) junction.

FIG. 37 is a sectional view of an embodiment of the invention for treatment of gastroparesis. Two anchor rings are attached to the device. A rigid cylinder is located in between the two anchors rings. An anti-reflux valve is located within the rigid cylinder. The anti-reflux valve allows for free flow of the stomach contents in to the duodenum, but can reduce the back flow of duodenal contents into the stomach from the duodenum. The device either does not utilize an intestinal bypass sleeve or it is short in length.

FIG. 38 is a sectional view of an embodiment of the invention implanted into a pylorus and duodenal bulb and duodenum. The rings are sized sufficiently large in diameter to engage the wall of the stomach pyloric antrum and the duodenal bulb. The central portion of the device is constructed of a rigid fixed-diameter cylinder. A needle, suture, T-bar, hollow helical anchor or screw type anchor is inserted into and or through the tissue of the pylorus to provide additional anchoring and securement of the intestinal bypass sleeve anchoring device to the gastro-intestinal anatomy.

FIG. 39 is a sectional view of an embodiment of the invention implanted into a pylorus and duodenal bulb and duodenum. The anchoring device is comprised of two disk-shaped inflatable balloons that are connected by a central cylinder. The balloons may be filled with a gas such as air or CO2, a liquid such as saline or polyethylene glycol or with a liquid polymer such as silicone, polyurethane or epoxy that cures into a gel or solid polymer after the balloon is filled. The central cylinder is either fixed in diameter or its diameter can be reduced during delivery of the device to the implant location in the human body. The diameter of the central cylinder will then elastically recover to its original diameter after it is released from the delivery catheter.

FIG. 40 is a sectional view of an embodiment of the invention implanted into a pylorus and duodenal bulb and duodenum. The rings are sized sufficiently large in diameter to engage the wall of the stomach pyloric antrum and the duodenal bulb. Magnets are attached to anchors rings. The magnets are attached to the anchor rings so that the polarity of the magnets is such that the magnets on the opposite sides of the pylorus are attracted to each other and exert a compression force to the pylorus.

FIG. 41 is a sectional view of an embodiment of the invention implanted into a pylorus and duodenal bulb and duodenum. An anchor ring is sized sufficiently large in diameter to engage the wall of the stomach pyloric antrum. The central portion of the device is constructed of a rigid fixed-diameter cylinder or a compressible cylinder. A needle, suture, T-bar, hollow helical anchor or screw type anchor is inserted into and or through the tissue of the pylorus to provide additional anchoring and securement of the intestinal bypass sleeve anchoring device to the gastro-intestinal anatomy.

While the invention is amenable to various modifications and alternative forms, specific embodiments have been shown by way of example in the views and are described in detail below. The intention, however, is not to limit the invention to the particular embodiments described. On the contrary, the invention is intended to cover all modifications, equivalents, and alternatives falling within the scope of the invention as defined by the appended claims.

### DETAILED DESCRIPTION

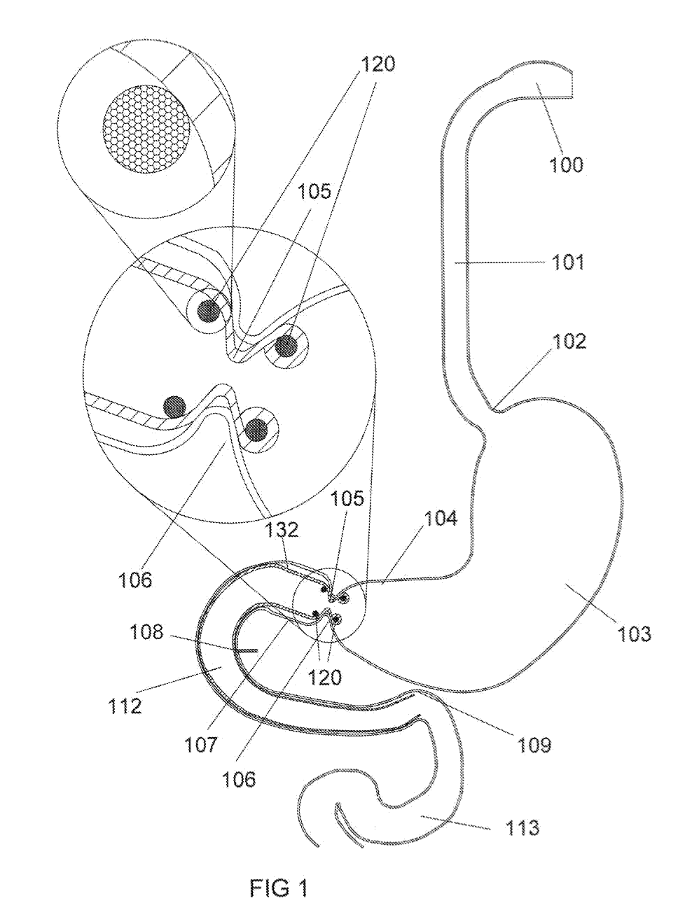

FIG. 1 is a sectional view of an embodiment of the invention implanted in a portion of a human digestive tract. As a person ingests food, the food enters the mouth 100, is chewed, and then proceeds down the esophagus 101 to the lower esophageal sphincter at the gastro-esophageal junction 102 and into the stomach 103. The food mixes with enzymes in the mouth 100 and in the stomach 103. The stomach 103 converts the food to a semi-fluid substance called chyme. The chyme enters the pyloric antrum 104 and exits the stomach 103 through the pylorus 106 and pyloric orifice 105. The small intestine is about 21 feet long in adults. The small intestine is comprised of three sections. The duodenum 112, jejunum 113 and ileum (not shown). The duodenum 112 is the first portion of the small intestine and is typically 10-12 inches long. The duodenum 112 is comprised of four sections: the superior, descending, horizontal and ascending. The duodenum 112 ends at the ligament of treitz 109. The papilla of vater 108 is the duct that delivers bile and pancreatic enzymes to the duodenum 112. The duodenal bulb 107 is the portion of the duodenum which is closest to the stomach 103.

As shown in FIG. 1, an intestinal bypass sleeve 132 is implanted in the duodenum 112 from the pylorus 106 to the ligament of treitz 109 or into the jejunum. The sleeve 132 is held in place at the pylorus 106 by two expandable anchors 120 one each side of the pylorus 106. These expandable anchors 120 may have a variety of sizes, shapes, and configurations as further described below. Further, in various embodiments of the invention, and as further described below, the anchors are connected or coupled by a variety of structures adapted to extend through the pylorus 106 and the pyloric orifice 105. Some exemplary expandable anchors 120 and some exemplary structures for connecting or coupling the expandable anchors 120 are disclosed for example in U.S. Pat. No. 5,820,584 and U.S. Pat. No. 7,122,058, each of which are incorporated herein by reference. In various disclosed embodiments, as further described below, the expandable anchors 120 are amendable to folding, bending, rolling, compressing, or otherwise collapsing to a reduced diameter configuration for delivery to and through the pylorus 106. Then, after delivery to the desired implantation site, and release from a delivery instrument or tool, the expandable anchors (or rings) 120 expand (or are expandable) to a post-implant configuration adapted for anchoring, or otherwise resisting migration, of the device within the gastrointestinal system.

In various exemplary embodiments, the sleeve 132 is integrally formed with or coupled to the expandable anchors 120. According to other exemplary embodiments, the sleeve 132 is removably or releasably coupled to the expandable anchors 120. According to various embodiments, the bypass sleeve has an anchor of between about 10 mm and about 35 mm. According to various embodiments, the bypass sleeve has a thickness of between about 0.001 and about 0.015 inches. Exemplary structures for removably or releasably coupling or attaching the sleeve 132 to the expandable anchors 120 are disclosed for example in U.S. patent application Ser. No. 12/752,697, filed Apr. 1, 2010, entitled “Modular Gastrointestinal Prostheses,” which is incorporated herein by reference. According to various embodiments, the sleeve 132 or the expandable anchors 120 (or both) are further coupled at the pylorus 106 using one or more of the techniques described in either of U.S. patent application Ser. No. 12/752,697 or U.S. patent application Ser. No. 12/833,605, filed Jul. 9, 2010, entitled “External Anchoring Configuration for Modular Gastrointestinal Prostheses,” both of which are incorporated herein by reference. According to various embodiments of the invention, the sleeve 132 may be configured and coupled to the expandable anchors 120, using one or more of the configurations disclosed in U.S. patent application Ser. No. 12/986,268, filed Jan. 7, 2011, entitled “Gastrointestinal Prostheses Having Partial Bypass Configurations,” which is incorporated herein by reference.

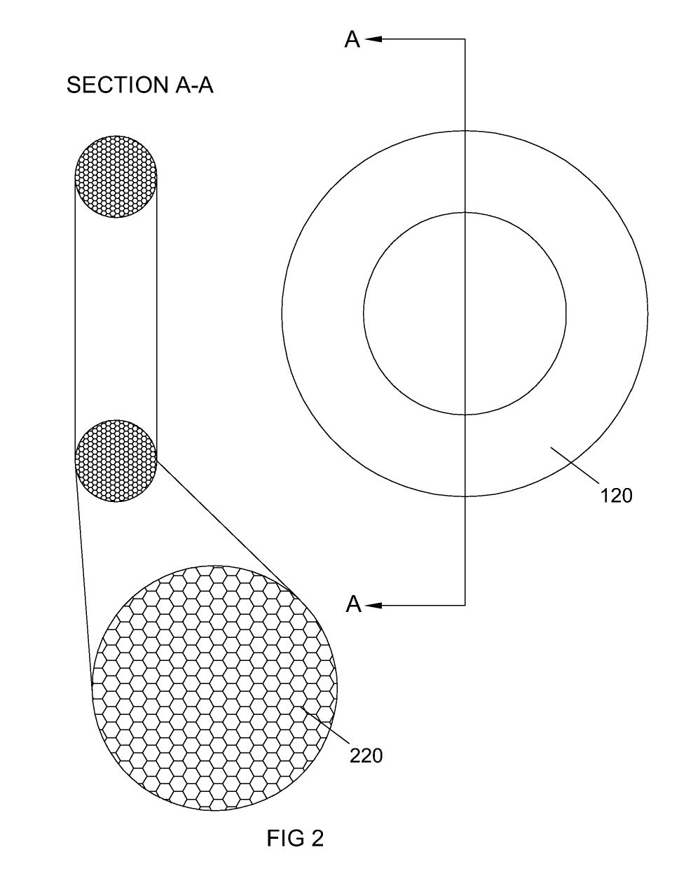

FIG. 2 shows an embodiment of an expandable anchor 120 adapted to anchor the intestinal bypass sleeve at the pylorus. In an exemplary embodiment, the expandable anchor is comprised of stranded wire. The stranded wire may be comprised of small diameter individual wires 220 or may be made up of small cables or bundles of braided wires. The wire diameter, in various exemplary embodiments, has a diameter in the 0.001 inch to 0.005 inch range. In other exemplary embodiments, the wire diameter may range from 0.0005 inch diameter up to 0.010 inch diameter. In various exemplary embodiments, the wire count ranges from 50 to 250 wires. In other embodiments, the wire count in the anchor ring may range from as few as 5 wires up to 5000 wires. In exemplary embodiments, the diameter of the expanded anchor 120 may range from 10 mm up to 60 mm in diameter. While some embodiments are made from stranded wire, in other embodiments, the anchor 120 is constructed from a single non-stranded wire.

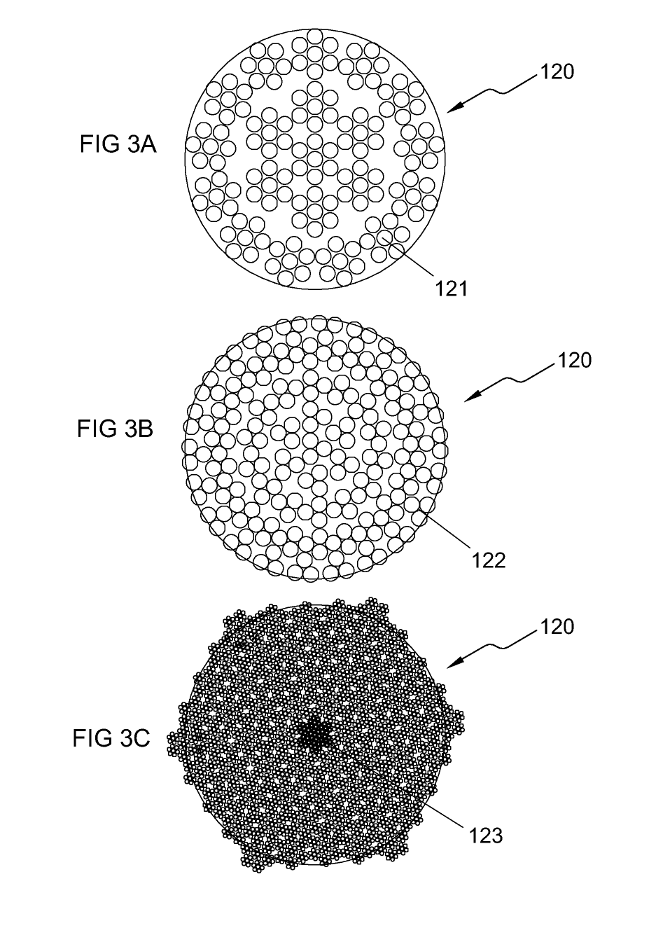

FIG. 3A shows a cross-sectional view of an embodiment of the expandable anchor 120 that can be used to anchor the intestinal bypass sleeve at the pylorus. The ring is wound from 1×7 Nitinol stranded cable. Each anchor ring 120 cross section has 20 wraps of 1×7 cable, with seven wire ends 121. Each anchor ring has a total number of 140 wires. The overall diameter of the anchor ring is in the 10 to 60 mm diameter range. The individual wire diameter, according to exemplary embodiments, is in the range of 0.001 to 0.005 inch diameter. The stranded ring can also be comprised of individual small diameter wires of a high wire count. Alternative stranded cable configurations may include 1×3, 1×7, 1×19, 7×7, 7×19 or 19×7. The stranded wires can also be of a braided configuration. The stranded wire may be made of Nitinol, stainless steel, L605, MP35N or Elgiloy, Peek or other suitable material.

FIG. 3B shows a cross-sectional view of an embodiment of expandable anchor 120 that can be used to anchor the intestinal bypass sleeve at the pylorus. The ring is wound from 1×3 Nitinol stranded cable 122. Each anchor 120 cross section has 65 wraps of 1×3 cable with three wire ends. Each anchor ring has a total number of 195 wires. The overall diameter of the anchor ring 120, in various embodiments, is in the 15 to 30 mm diameter range. The individual wire diameter, in exemplary embodiments, is in the range of 0.001 to 0.005 inch.

FIG. 3C shows a cross-sectional view of an embodiment of expandable anchor 120 that can be used to anchor the intestinal bypass sleeve at the pylorus. The ring is wound from 1×3 Nitinol stranded cable 123. Each anchor 120 cross section has 60 wraps of 7×7 cable with forty-nine wire ends. Each anchor ring has a total number of 2940 wires. The overall diameter of the anchor ring, in various embodiments, is in the 15 to 30 mm diameter range. The individual wire diameter, in exemplary embodiments, is in the range of 0.0005 to 0.002 inch.

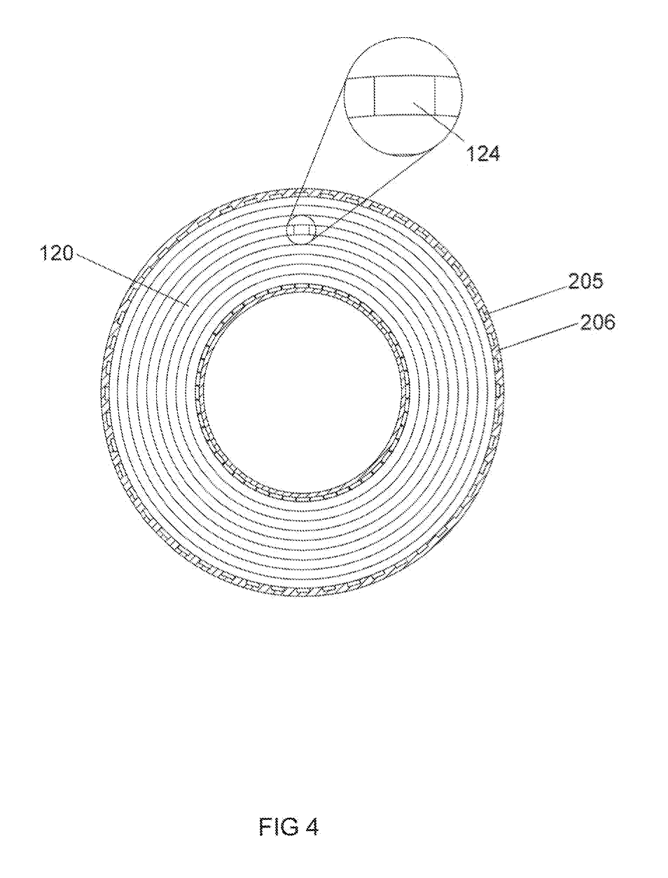

FIG. 4 is a cross-sectional view of an expandable anchor 120 that can be used to anchor the intestinal bypass sleeve at the pylorus. The expandable anchor 120 is formed by wrapping stranded wire around a fixed-diameter mandrel. The wrapped stranded wire has a starting end and a finishing end. The two ends may be joined together by placing a crimp sleeve 124 over the two cable ends and mechanically crimping the diameter of the sleeve to mechanically hold the two free ends together. Other mechanical joining means may be used or bonding by adhesive, laser welding or soldering. The expandable anchor 120 may also have an external wire wrap 205 coiled around the bundle of wires that form the expandable anchor. The wire wrap 205 may be made from Nitinol, stainless steel or MP35N, L605 or Elgiloy. A polymer covering 206 may encapsulate the expandable anchor 120 and the wire wrap 205. The polymer covering may be comprised of silicone, polyurethane, ePTFE, FEP, PTFE, polyethylene or other suitable polymer. The polymer covering 206 provides a structure to secure the items 120 and 205 together. It also adds mechanical strength to the anchor ring 120 as the elasticity of the polymer provides an additive radial strength or force. It also provides for a polymer covering 206 in which a drug may be loaded and eluted out of the polymer covering 206 to provide a therapeutic benefit.

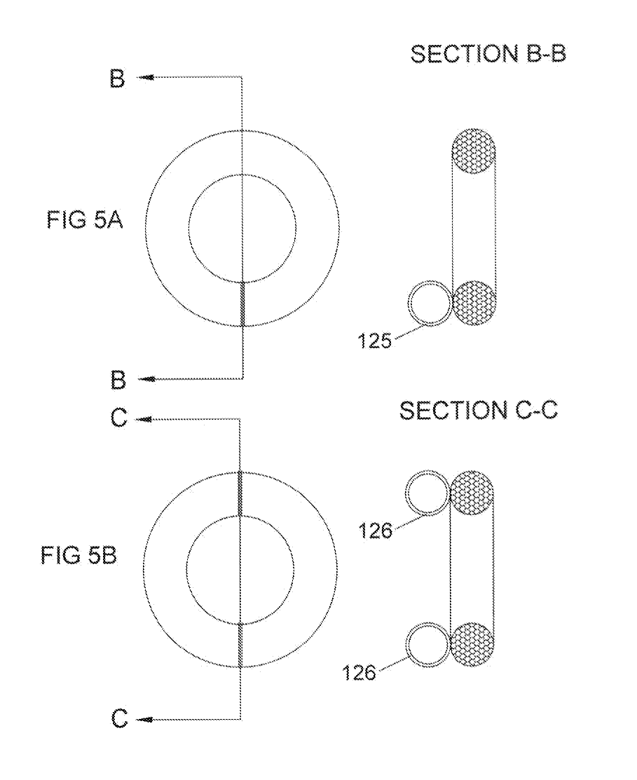

FIG. 5A shows an alternative embodiment of expandable anchor that can be used to anchor the intestinal bypass sleeve at the pylorus. The anchor has an integral retrieval loop 125 that can be grabbed to recover or remove the device. FIG. 5B is a view of an alternative embodiment of expandable anchor that can be used to anchor the intestinal bypass sleeve at the pylorus. The anchor has two integral retrieval loops 126 that can be grabbed to recover or remove the device.

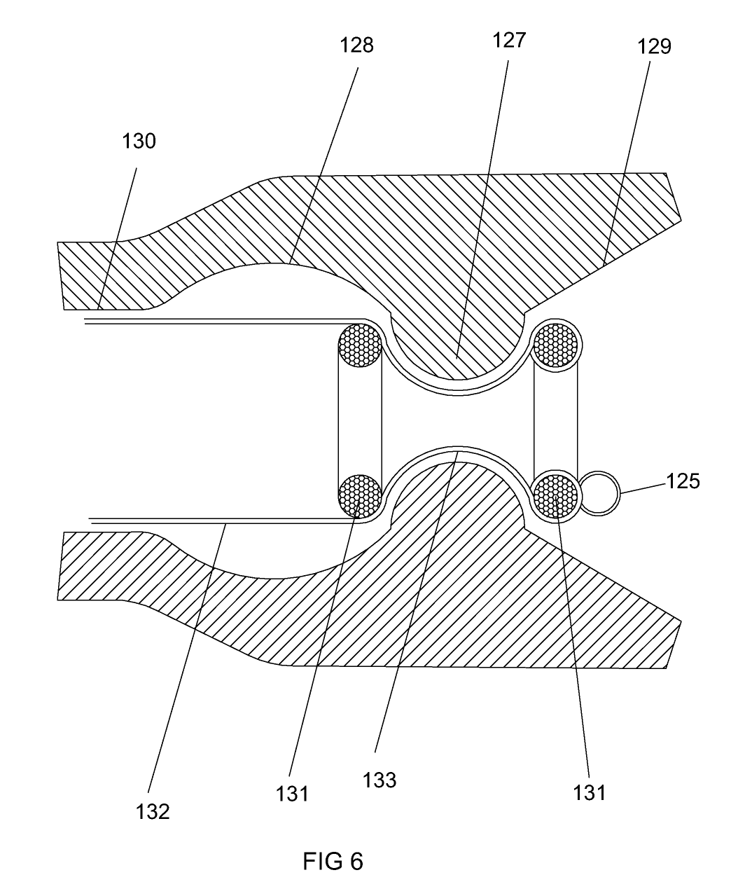

FIG. 6 is a cross-sectional view of an embodiment of the invention implanted into a pylorus 127, a duodenal bulb 128, and a duodenum 130. The rings 131, in exemplary embodiments, are sized sufficiently small in diameter that there is minimal to no contact force between the rings and the stomach pyloric antrum 129. Likewise, in various embodiments, the rings 131 are sized sufficiently small in diameter that there is little or no contact between the rings and the duodenal bulb 128. The anchor rings 131 are sufficiently large in diameter that they are larger than the maximum opened diameter of the pylorus, and thereby provide for an anchoring mechanism to hold the intestinal bypass sleeve 132 in place or otherwise resist migration. A drug coating may be applied to the inside or outside surface of the sleeve 132. A drug may be incorporated within the wall thickness of the sleeve 132. The drug may be eluted from the sleeve 132. The intestinal bypass sleeve 132 can vary in length from 1-2 inches in length up to several feet. In some embodiments, the sleeve extends along (i.e., bypasses) the length of the duodenum up to the ligament of treitz. In other embodiment, the sleeve is longer and bypasses into the jejunum. The pyloric portion of the device 133 disposed between the two anchor rings can be made from a flexible polymer material such a silicone, polyurethane, polyethylene or polytetrafluoroethylene (PTFE) or expanded PTFE. A drug coating may be applied to the inside or outside surface of the pyloric portion 133. A drug may be incorporated within the wall thickness of the pyloric portion 133. The drug may be eluted from the pyloric portion 133. In various embodiments, the pyloric portion has a width equal to or greater than a width of the pylorus.

The diameter and length of the pyloric portion 133 of the device, according to some embodiments, is sized and configured to allow normal opening and closing of the pylorus. In other embodiments, the diameter and length of the pyloric portion 133 is sized and configured to allow for partial closing of the pylorus or to fully hold the pylorus open. An optional retrieval loop 125 allows for easier capturing and removal of the device from the human body.

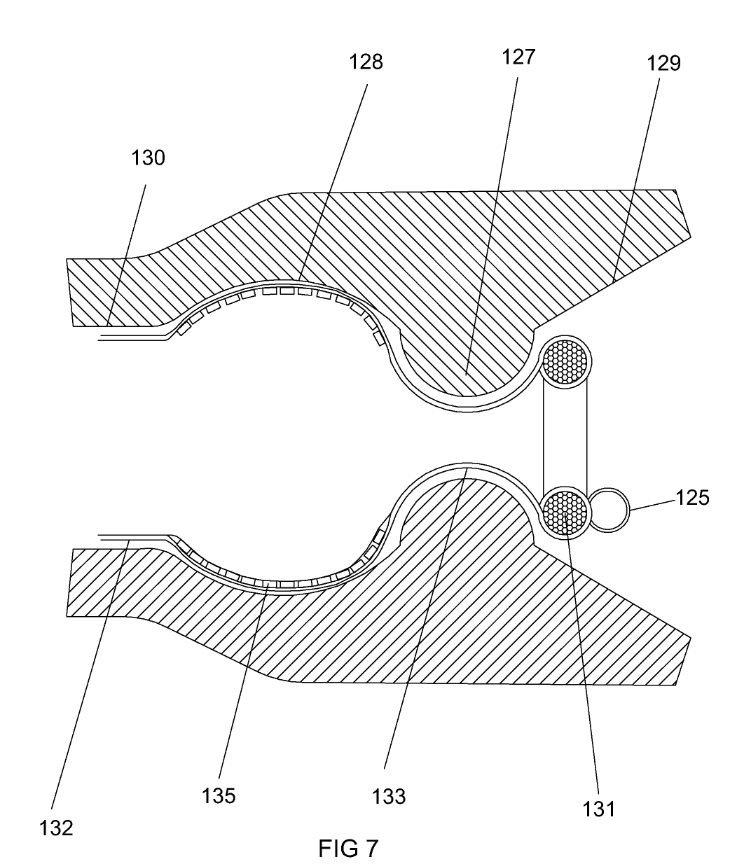

FIG. 7 is a cross-sectional view of an embodiment of the invention implanted into a pylorus 127, a duodenal bulb 128, and a duodenum 130. As shown, the device includes a ring-like structure 131 is on the stomach side of the pylorus 127 and a conventional barrel-shaped stent 135 is on the duodenal side of the pylorus 127. The barrel-shaped stent 135 may be of a slotted tubular design (cut from a piece of tubing), formed from wire or may be made from a wound coil. The stent 135 may be self expanding or balloon expandable. The rings 131 are sized sufficiently small in diameter that there is minimal to no contact force between the ring and the stomach pyloric antrum 129. The anchor ring 131 and stent 135 are larger in diameter than the maximum opened diameter of the pylorus and therefore provide for an anchoring mechanism to hold the intestinal bypass sleeve 132 or otherwise resist migration. The intestinal bypass sleeve 132 can vary in length from 1-2 inches in length up to several feet. In some embodiments, the sleeve bypasses the length of the duodenum up to the ligament of treitz. The sleeve can be longer and bypass into the jejunum. The pyloric portion of the device 133 in between the two anchor rings can be made from a flexible polymer material such a silicone, polyurethane, polyethylene or polytetraflouorethylene (PTFE) or expanded PTFE or other suitable polymer. The diameter and length of the pyloric portion 133 of the device can be sized to allow normal opening and closing of the pylorus or it can allow for partial closing of the pylorus, or it may also fully hold the pylorus open. A retrieval loop 125 allows for easier capturing and removal of the device from the human body.

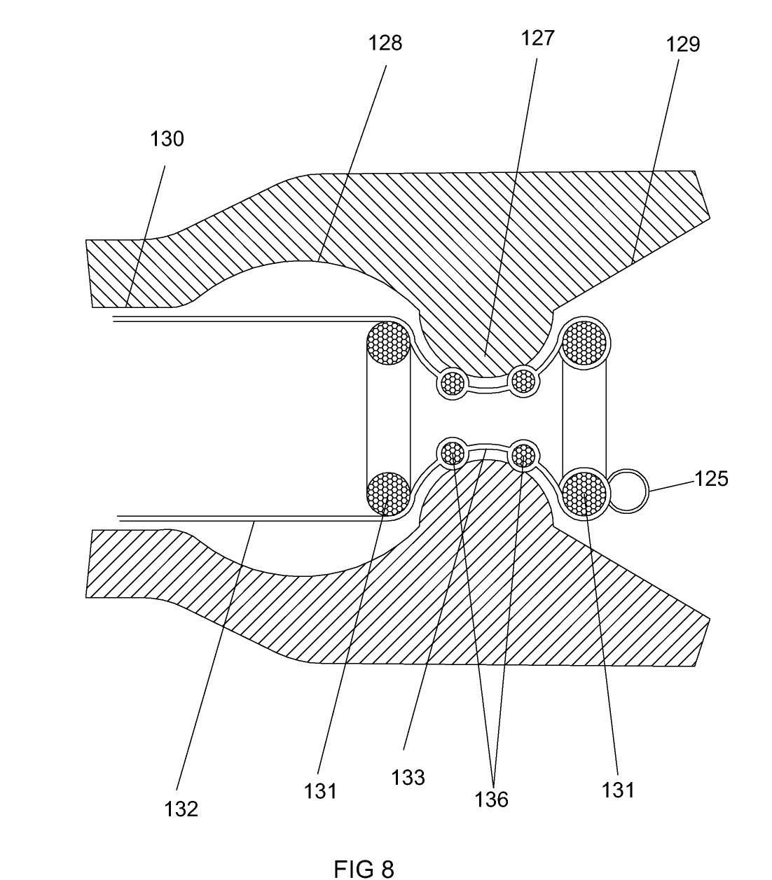

FIG. 8 is a cross-sectional view of an embodiment of the invention implanted into a pylorus 127, a duodenal bulb 128, and a duodenum 130. The rings can be sized sufficiently small in diameter that there is minimal to no contact force between the rings and the stomach pyloric antrum 129 and the duodenal bulb 128. The anchor rings 131 are sufficiently large in diameter that they are larger than the maximum opened diameter of the pylorus and therefore provide for an anchoring mechanism to hold the intestinal bypass sleeve 132. The intestinal bypass sleeve can vary in length from 1-2 inches in length up to several feet. In some embodiments, the sleeve bypasses the length of the duodenum up to the ligament of treitz. The sleeve can be longer and bypass into the jejunum. The pyloric portion of the device 133 in between the two anchor rings can be made from a flexible material such a silicone, polyurethane, polyethylene or polytetraflouorethylene (PTFE) or expanded PTFE. The diameter and length of the pyloric portion 133 of the device can be sized to allow normal opening and closing of the pylorus or it can allow for partial closing of the pylorus, or it may also fully hold the pylorus open. Two smaller diameter ring-like structures 136 reside within the area of the pylorus opening to provide for increased rigidity of the pyloric portion of the device to prevent the pylorus from closing fully. A retrieval loop 125 allows for easier capturing and removal of the device from the human body.

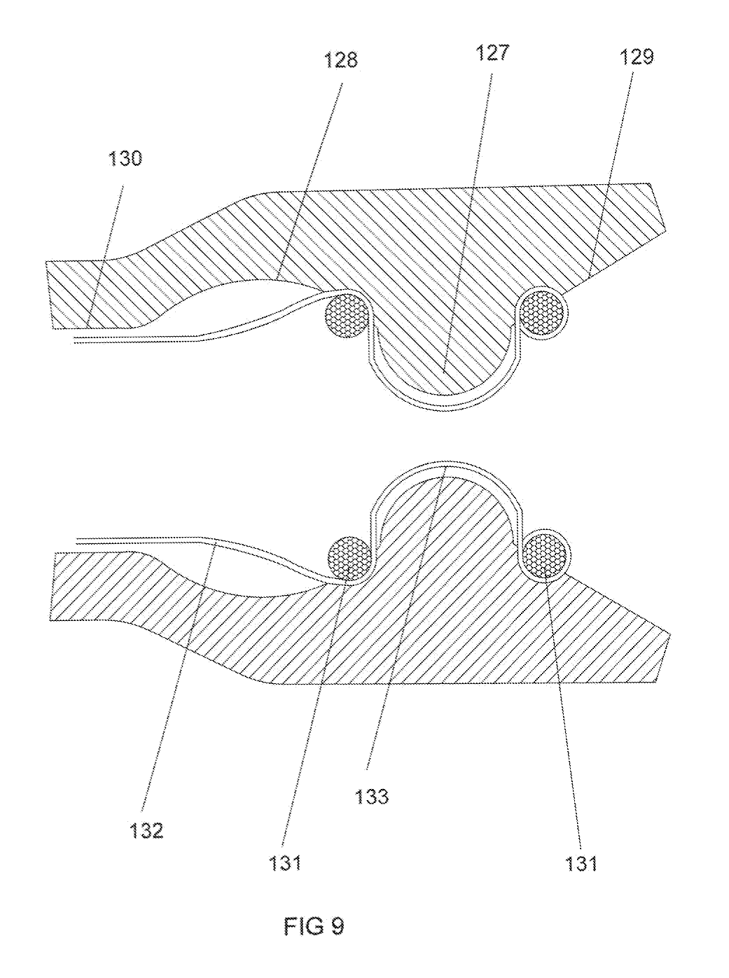

FIG. 9 is a cross-sectional view of an embodiment of the invention implanted into a pylorus 127, a duodenal bulb 128, and a duodenum 130. The rings are sized sufficiently large in diameter that there is a contact force between the ring diameter and the stomach pyloric antrum 129 and the duodenal bulb 128. The anchor rings 131 are sufficiently large in diameter that they are larger than the maximum opened diameter of the pylorus and therefore provide an anchoring mechanism to hold the intestinal bypass sleeve 132. The intestinal bypass sleeve can vary in length from 1-2 inches in length up to several feet. In a preferred embodiment, the sleeve bypasses the length of the duodenum up to the ligament of treitz. The sleeve can also be longer and bypass into the jejunum. The pyloric portion of the device 133 in between the two anchor rings can be made from a flexible material such a silicone, polyurethane, polyethylene or polytetraflouorethylene (PTFE) or expanded PTFE (ePTFE). The diameter and length of the pyloric portion 133 of the device can be sized to allow normal opening and closing of the pylorus or it can allow for partial closing of the pylorus, or it may also fully hold the pylorus open.

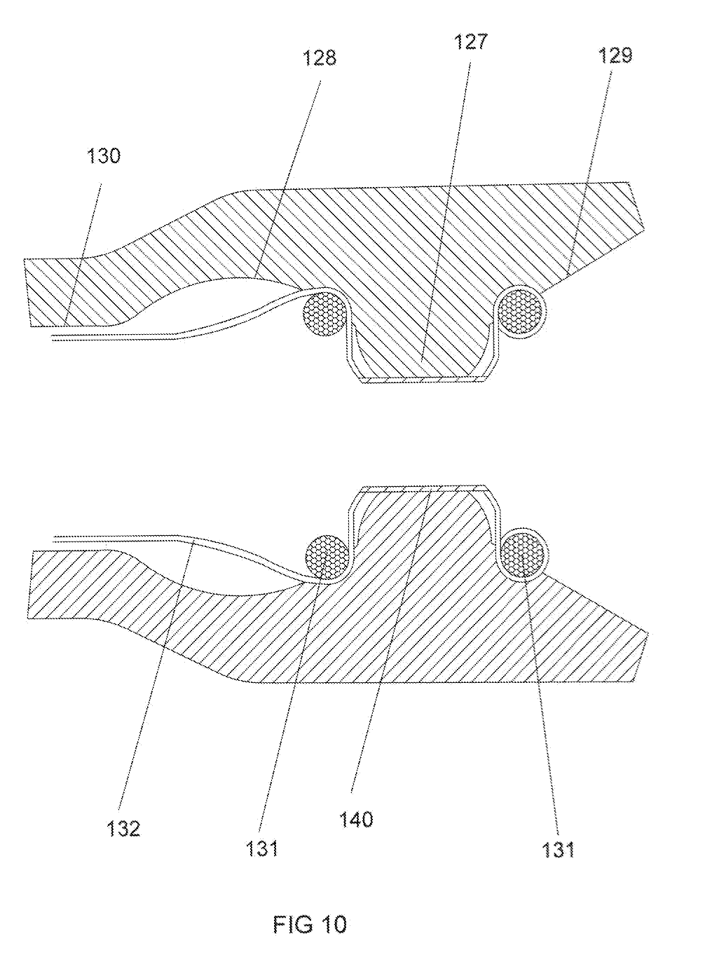

FIG. 10 is a cross-sectional view of an embodiment of the invention implanted into a pylorus 127, a duodenal bulb 128, and a duodenum 130. The rings are sized sufficiently large in diameter that there is a contact force between the ring diameter and the stomach pyloric antrum 129 and the duodenal bulb 128. The anchor rings 131 are sufficiently large in diameter that they are larger than the maximum opened diameter of the pylorus and therefore provide an anchoring mechanism to hold the intestinal bypass sleeve 132. The intestinal bypass sleeve can vary in length from 1-2 inches in length up to several feet. In a preferred embodiment, the sleeve bypasses the length of the duodenum up to the ligament of treitz. The sleeve can also be longer and bypass into the jejunum. The pyloric portion 140 located between the two anchor rings can be made from a rigid (or semi-rigid) cylinder made from plastic material such as delrin, peek, high density polyethylene, polycarbonate or other suitable polymer. The pyloric portion 140 may also be made from stainless steel, titanium or Nitinol. The fixed diameter of the pyloric portion 140 of the device can be sized to provide for a full opening of the pylorus and not allow the pylorus to close normally. In various embodiments, the diameter of the pyloric portion 140 ranges from as about 3 mm to about 15 mm. In various embodiments, the pyloric portion 140 has sufficient stiffness or rigidity to prevent (or substantially prevent) radial movement (i.e. twisting) of the anchor rings 131 with respect to each other. In various embodiments, the pyloric portion 140 has sufficient stiffness or rigidity to maintain (or substantially maintain) the anchor rings 131 in desired (e.g., parallel) planes with respect to each other. These embodiments may help maintain the device securely in place at the pylorus and also may help ensure that the pylorus remains fully or partially open.

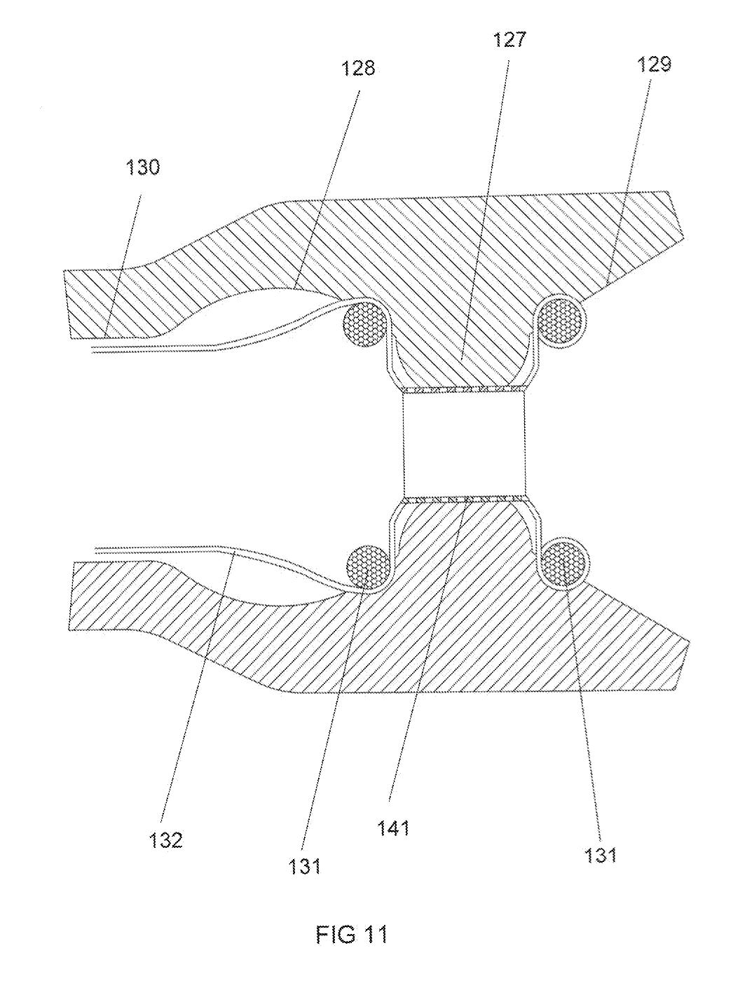

FIG. 11 is a cross-sectional view of an embodiment of the invention implanted into a pylorus 127, a duodenal bulb 128, and a duodenum 130. The rings are sized sufficiently large in diameter that there is a contact force between the ring diameter and the stomach pyloric antrum 129 and the duodenal bulb 128. The anchor rings 131 are sufficiently large in diameter that they are larger than the maximum opened diameter of the pylorus and therefore provide an anchoring mechanism to hold the intestinal bypass sleeve 132. The intestinal bypass sleeve can vary in length from a 1-2 inches in length up to several feet. In some embodiments, the sleeve bypasses the length of the duodenum up to the ligament of treitz. The sleeve can also be longer and bypass into the jejunum. The pyloric (or central) portion 141 of the device disposed between the two anchor rings is constructed of an expandable diameter cylinder constructed of a stent-like construction. The cylinder has enough radial strength to resist compression by the pylorus when it closes, but provides for a central portion which can be compressed in diameter when it is loaded onto a delivery catheter so that the catheter diameter may be smaller. The pyloric portion 141 may be self expanding or may require the use of a balloon to expand it. The pyloric portion 141 may also be initially implanted at an initial diameter and then the diameter may be adjusted in size made larger or smaller if required because there is inadequate weight loss or a dumping syndrome in which the food moves too rapidly into the duodenum. According to various embodiments, the diameter of the pyloric portion 141 may range from 3 mm to 18 mm. In various embodiments, the pyloric portion 141 has sufficient stiffness or rigidity to prevent (or substantially prevent) radial movement (i.e. twisting) of the anchor rings 131 with respect to each other. In various embodiments, the pyloric portion 141 has sufficient stiffness or rigidity to maintain (or substantially maintain) the anchor rings 131 in desired (e.g., parallel) planes with respect to each other. These embodiments may help maintain the device securely in place at the pylorus and also may help ensure that the pylorus remains fully or partially open.

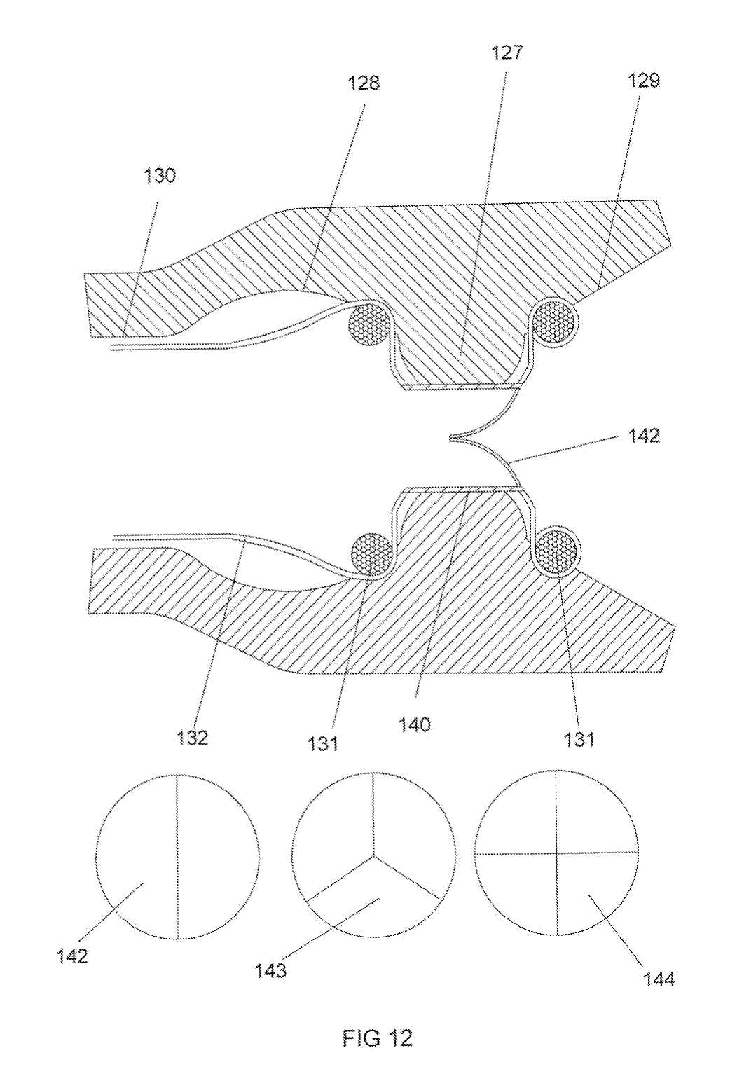

FIG. 12 is a cross-sectional view of an embodiment of the invention implanted into a pylorus 127, a duodenal bulb 128, and a duodenum 130. The rings are sized sufficiently large in diameter that there is a contact force between the ring diameter and the stomach pyloric antrum 129 and the duodenal bulb 128. The anchor rings 131 are sufficiently large in diameter that they are larger than the maximum opened diameter of the pylorus and therefore provide an anchoring mechanism to hold the intestinal bypass sleeve 132. The intestinal bypass sleeve can vary in length from a couple of inches in length up to several feet. In some embodiments, the sleeve bypasses the length of the duodenum up to the ligament of treitz. The sleeve can also be longer and bypass into the jejunum. The pyloric portion of the device 140 in between the two anchor rings can be made from a rigid cylinder made from plastic material such as delrin, peek, high density polyethylene, polycarbonate or other suitable polymer. The pyloric portion may also be made from stainless steel, titanium or Nitinol. The fixed diameter of the pyloric portion 140 of the device can be sized to provide for a full opening of the pylorus and not allow the pylorus to close normally. The diameter of the pyloric portion 140 could range from as small as 3 mm to as large as 16 mm.

In various embodiments, the central lumen of device includes a one-way anti-reflux valve 142. The anti-reflux valve 142 allows for unobstructed flow in the direction of the stomach antrum 129 to the pylorus 127, but limits flow in the reverse direction. Similarly, the anti-reflux valve 142 may prevent (or limit) retrograde (i.e., from the duodenum to the stomach antrum) movement (i.e., eversion) of the sleeve. The pyloric portion 140 may also incorporate a stoma that be used to restrict flow. The stoma may be configured to be a round hole in a polymer membrane disk which is located in the pyloric portion 140 of the device. The stoma may be constructed from a polymer. The inside diameter of the stoma may range from 3 mm to 10 mm. The anti-reflux valve 142 can be constructed of a duck bill design with two flexible leaflets, or may utilize other designs such as a tri-leaflet valve 143 or quad-leaflet valve 144. The anti-reflux valve may be constructed of silicone or polyurethane, polyethylene, ePTFE or other suitable polymer.

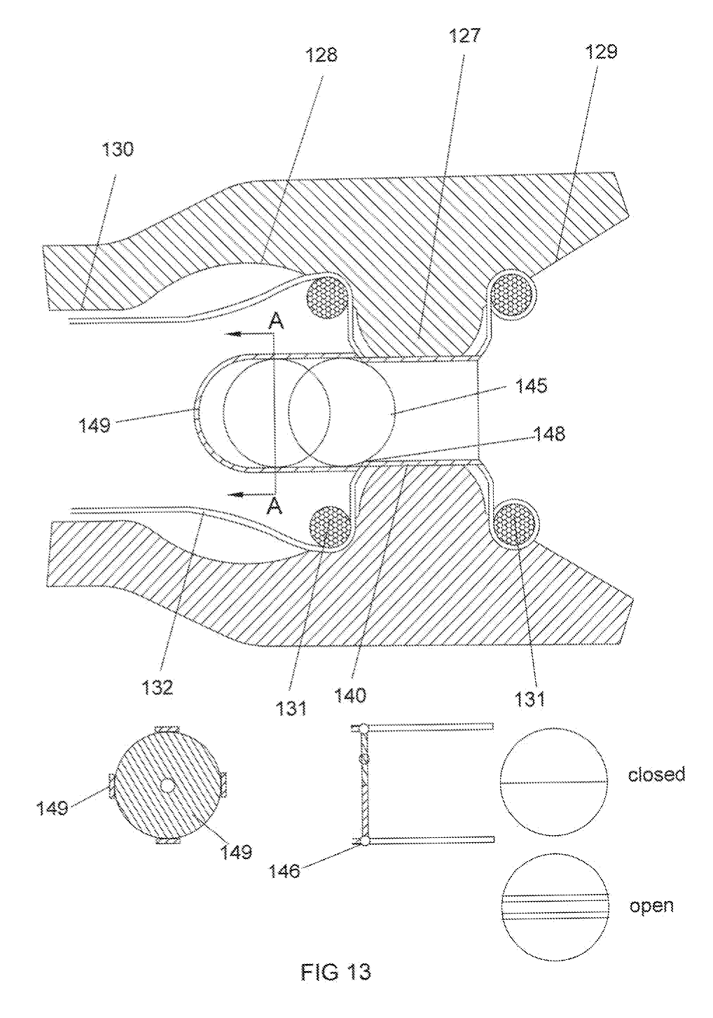

FIG. 13 is a cross-sectional view of an embodiment of the invention implanted into a pylorus 127, a duodenal bulb 128, and a duodenum 130. The rings are sized sufficiently large in diameter that there is a contact force between the ring diameter and the stomach pyloric antrum 129 and the duodenal bulb 128. The anchor rings 131 are sufficiently large in diameter that they are larger than the maximum opened diameter of the pylorus and therefore provide an anchoring mechanism to hold the intestinal bypass sleeve 132. The intestinal bypass sleeve can vary in length from a couple of inches in length up to several feet. In some embodiments, the sleeve bypasses the length of the duodenum up to the ligament of treitz. The sleeve can also be longer and bypass into the jejunum. The pyloric portion of the device 140 in between the two anchor rings can be made from a rigid cylinder made from plastic material such as delrin, peek, high density polyethylene, polycarbonate or other suitable polymer. The pyloric portion 140 may also be made from stainless steel, titanium or Nitinol. The fixed diameter of the pyloric portion 140 of the device can be sized to provide for a full opening of the pylorus and not allow the pylorus to close normally.

In various embodiments, the diameter of the pyloric portion 140 could range from about 3 mm to about 18 mm. In other embodiments, the diameter of the pyloric portion 140 could range from as about 4 mm to about 12 mm. As shown, the central lumen of the device has a one way anti-reflux valve 145. The anti-reflux valve 145 allows for unobstructed flow in the direction of the stomach antrum 129 to the pylorus 127, but limits flow in the reverse direction. The anti-reflux valve can be constructed of a ball and cage design 149. When the ball is all the way towards the cage 149, the valve is all the way open and allows flow of chyme from the stomach 129 to duodenum 130. When the ball is up against the valve seat 148 it is closed and the retrograde flow from the duodenum 130 to the stomach 129 should be minimized. The ball and cage may be constructed of metal or plastics. A bi-leaflet 146 valve may also be suitable for the reflux valve.

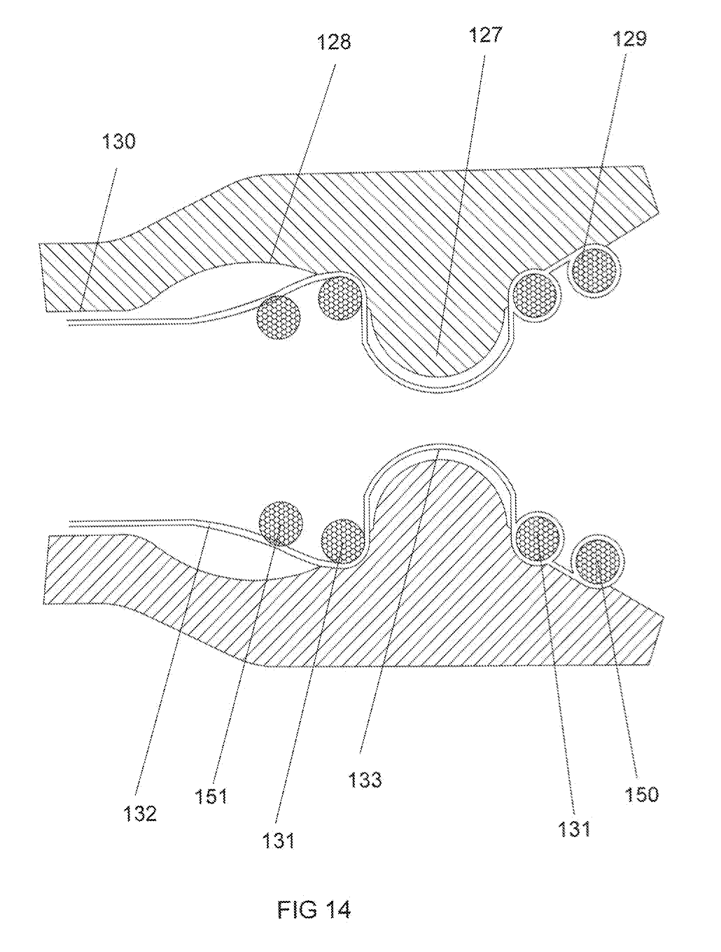

FIG. 14 is a cross-sectional view of an embodiment of the invention implanted into a pylorus 127, a duodenal bulb 128, and a duodenum 130. The rings are sized sufficiently large in diameter that there is a contact force between the ring diameter and the stomach pyloric antrum 129 and the duodenal bulb 128. The anchor rings 131 are sufficiently large in diameter that they are larger than the maximum opened diameter of the pylorus and therefore provide an anchoring mechanism to hold the intestinal bypass sleeve 132. The intestinal bypass sleeve can vary in length from a 1-2 inches in length up to several feet. In some embodiments, the sleeve bypasses the length of the duodenum up to the ligament of treitz. The sleeve can also be longer and bypass into the jejunum. The pyloric portion of the device 133 in between the two anchor rings can be made from a flexible material such a silicone, polyurethane, polyethylene or polytetraflouorethylene (PTFE) or expanded PTFE. The diameter and length of the pyloric portion 133 of the device can be sized to allow normal opening and closing of the pylorus or it can allow for partial closing of the pylorus, or it may also fully hold the pylorus open. Additional anchor ring(s) 150 can be added to the stomach antrum 129 side of the device if required for achieving a more funnel-shaped opening or to increase anchoring force. Additional anchor ring(s) 151 can be added to the duodenal bulb 128 side of the device to increase the anchoring force if required. In various embodiments, the pyloric portion 133 has sufficient stiffness or rigidity to prevent (or substantially prevent) radial movement (i.e. twisting) of the anchor rings 131 with respect to each other. In various embodiments, the pyloric portion 133 has sufficient stiffness or rigidity to maintain (or substantially maintain) the anchor rings 131 in desired (e.g., parallel) planes with respect to each other. These embodiments may help maintain the device securely in place at the pylorus and also may help ensure that the pylorus remains fully or partially open.

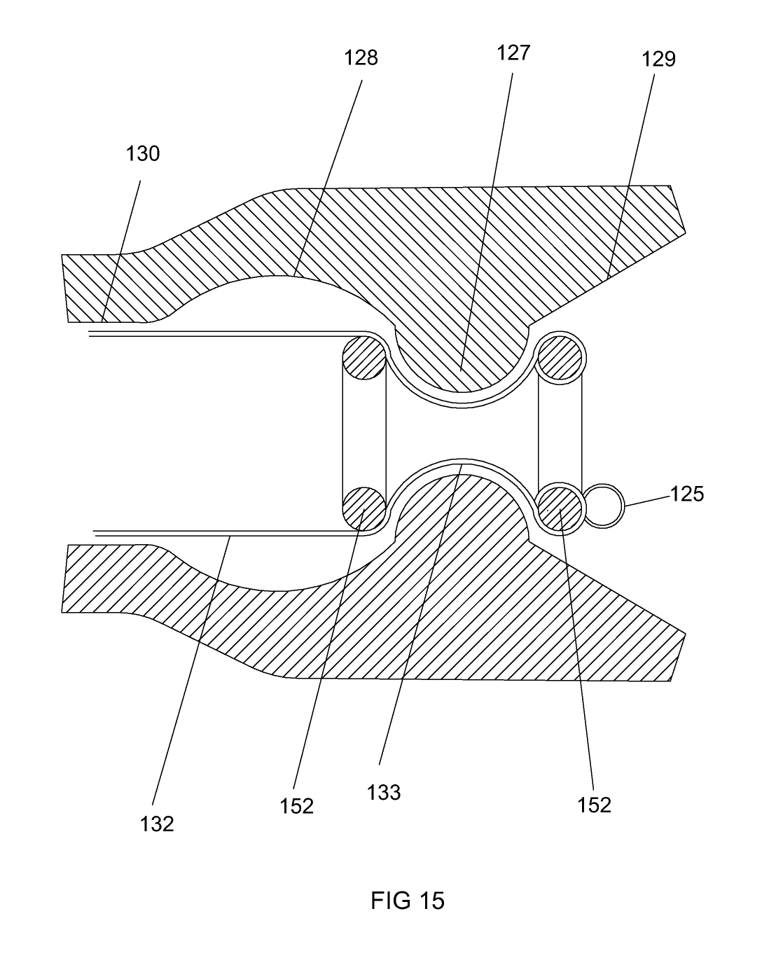

FIG. 15 is a cross-sectional view of an embodiment of the invention implanted into a pylorus 127, a duodenal bulb 128, and a duodenum 130. The anchor rings 152 are sized sufficiently small in diameter that there is minimal to no contact force between the rings and the stomach pyloric antrum 129 and the duodenal bulb 128. The anchor rings 152 are sufficiently large in diameter that they are larger than the maximum opened diameter of the pylorus and therefore provide for an anchoring mechanism to hold the intestinal bypass sleeve 132. The anchor rings 152 are comprised of an elastic polymer such as silicone, polyurethane or a fluoroelastomer such as Viton. The intestinal bypass sleeve can vary in length from a 1-2 inches in length up to several feet. In some embodiments, the sleeve bypasses the length of the Duodenum up to the ligament of treitz. The sleeve can be longer and bypass into the jejunum. The pyloric portion 133 between the two anchor rings can be made from a flexible material such a silicone, polyurethane, polyethylene or polytetraflouorethylene (PTFE) or expanded PTFE. The diameter and length of the pyloric portion 133 of the device can be sized to allow normal opening and closing of the pylorus or it can allow for partial closing of the pylorus, or it may also fully hold the pylorus open. A retrieval loop 125 allows for easier capturing and removal of the device from the human body.

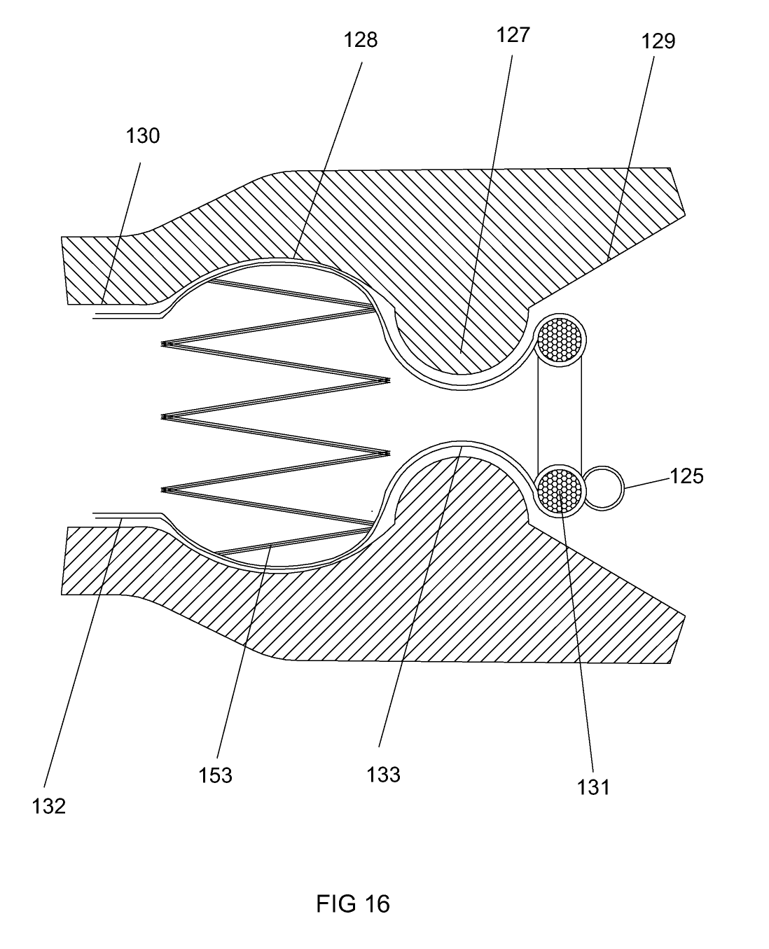

FIG. 16 is a cross-sectional view of an embodiment of the invention implanted into a pylorus 127, a duodenal bulb 128, and a duodenum 130. A ring-like structure is on the stomach side of the pylorus 127 and a conventional stent 153 is on the duodenal side of the pylorus. The conventional stent design 153 is formed from stranded or highly stranded wire. The ring 131 and stent 153 are sized sufficiently small in diameter that there is minimal to no contact force between the ring and the stomach pyloric antrum 129. The anchor ring 131 and stent 153 are sufficiently large in diameter that they are larger than the maximum opened diameter of the pylorus and therefore provide for an anchoring mechanism to hold the intestinal bypass sleeve 132. The intestinal bypass sleeve can vary in length from 1-2 inches in length up to several feet. In some embodiments, the sleeve bypasses the length of the duodenum up to the ligament of treitz. The sleeve can be longer and bypass into the jejunum. The pyloric portion of the device 133 in between the two anchor rings can be made from a flexible material such as silicone, polyurethane, polyethylene or polytetraflouorethylene (PTFE) or expanded PTFE or other suitable polymer. The diameter and length of the pyloric portion 133 of the device can be sized to allow normal opening and closing of the pylorus or it can allow for partial closing of the pylorus, or it may also fully hold the pylorus open. A retrieval loop 125 allows for easier capturing and removal of the device from the human body.

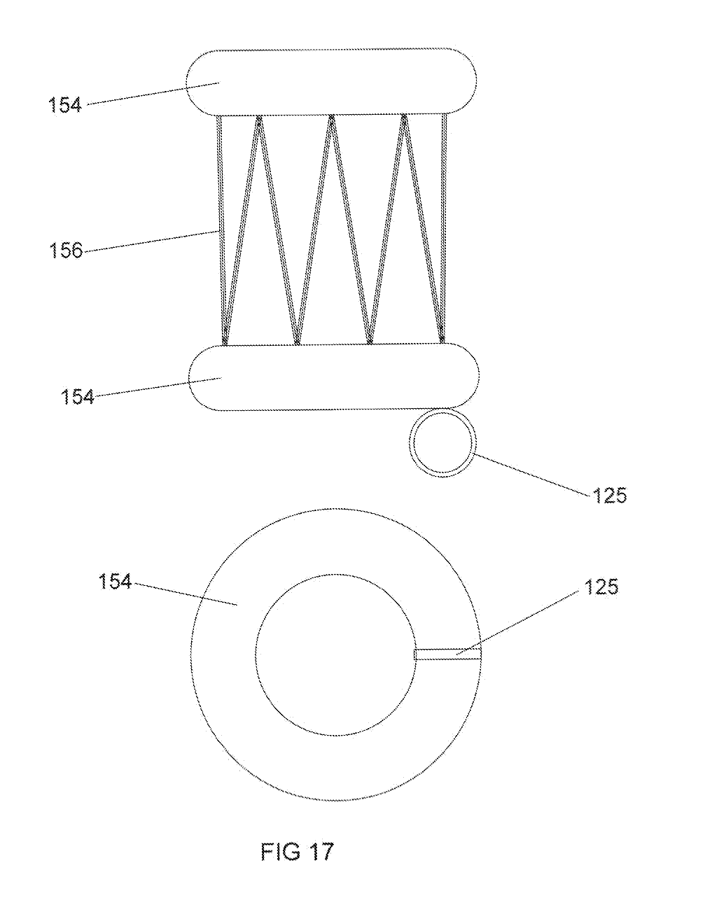

FIG. 17 shows an embodiment of the invention including two anchor rings 154 constructed of stranded Nitinol wire. The two rings are linked by longitudinal links 156. The longitudinal links 156 may be constructed of solid Nitinol wire or stranded Nitinol wire or stranded other material such as stainless steel, Elgiloy, MP35N or L605. The longitudinal links may extend parallel to the longitudinal axis of the device or may run at an angle to the longitudinal axis. The longitudinal links 156 may be constructed integral with the anchor rings 154, from the same wire and wound at the same time or they may be made discretely and simply attached to the anchor rings 154. A removal loop 125 is attached to one of the anchor rings 154. In various embodiments, the longitudinal links are sufficiently flexible to allow the pylorus to close (or partially close) when the device is implanted across the pylorus. In other embodiments, the longitudinal links are sufficiently stiff to maintain the pylorus in an open (or substantially open) configuration when the device is implanted across the pylorus.

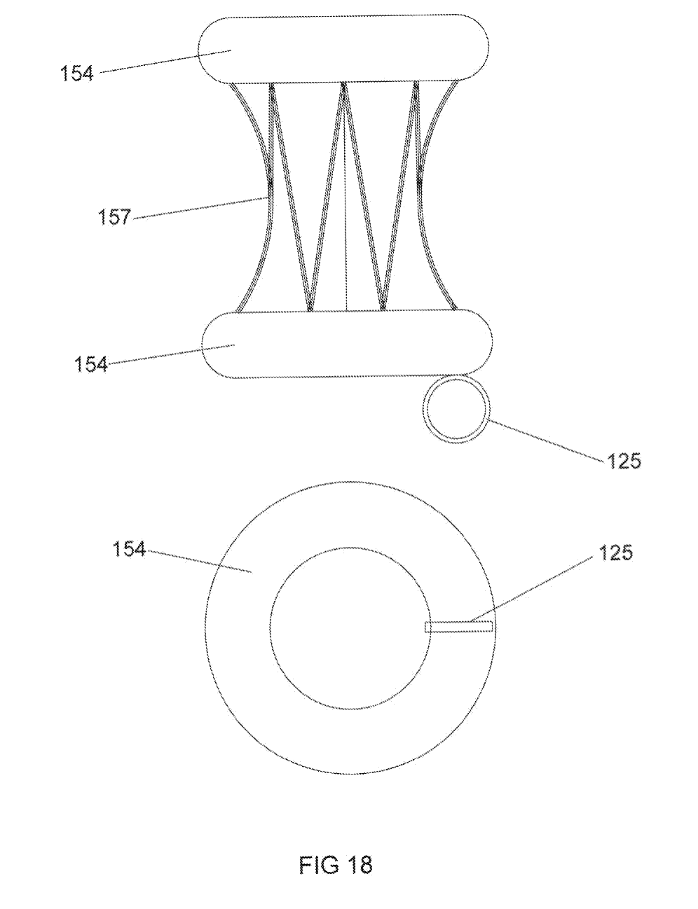

FIG. 18 shows an embodiment of the invention including two anchor rings 154 constructed of stranded Nitinol wire. The two anchor rings 154 rings are linked by longitudinal links 157, the longitudinal links 157 are curved to form a central portion which has a smaller diameter than the anchor rings 154. The longitudinal links 157 may be constructed of solid Nitinol wire or stranded Nitinol wire or stranded other material such as stainless steel, Elgiloy, MP35N or L605. The longitudinal links 157 may extend parallel to the longitudinal axis of the device or may run at an angle to the longitudinal axis. The longitudinal links are curved so that the central portion of the linked structure forms a narrow diameter (waist) in the middle between the two anchor rings 154. The waist area may be aligned with and straddle the opening of the pylorus. The longitudinal links 157 may be constructed integral with the anchor rings 154, from the same wire and wound at the same time or they may be made discretely and simply attached to the anchor rings. A removal loop is attached to one of the anchor rings 125.

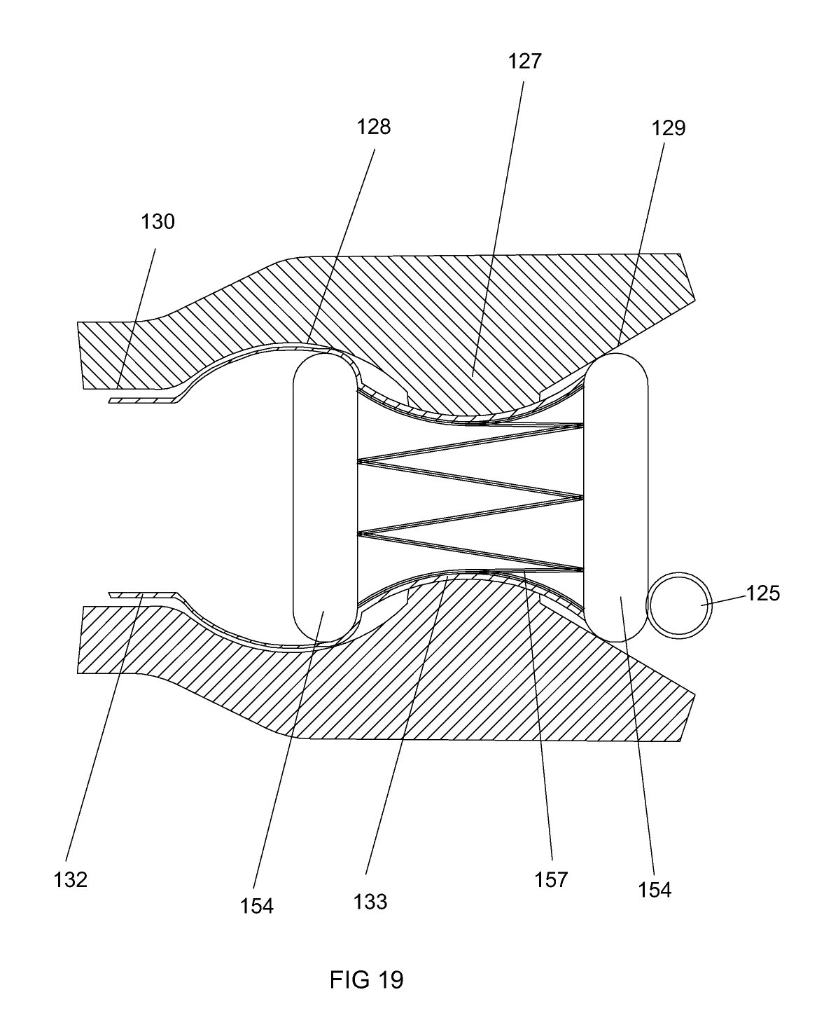

FIG. 19 is a sectional view of the anchor disclosed in FIG. 18 with a intestinal bypass sleeve 132 attached to the anchor ring 154 from the proximal end to distal end. The anchor and sleeve is implanted across the pylorus 127 from the stomach pyloric antrum 129 to the duodenal bulb 130, with one anchor ring 154 located on each side of the pylorus and a pyloric (or central) portion 133 extending between the anchor rings 154. The intestinal bypass sleeve 132 continues on into the duodenum 130 to the ligament of treitz or into the jejunum. In various embodiments, the pyloric portion 133 has sufficient stiffness or rigidity to prevent (or substantially prevent) radial movement (i.e. twisting) of the anchor rings 154 with respect to each other. In various embodiments, the pyloric portion 133 has sufficient stiffness or rigidity to maintain (or substantially maintain) the anchor rings 154 in desired (e.g., parallel) planes with respect to each other. These embodiments may help maintain the device securely in place at the pylorus and also may help ensure that the pylorus remains fully or partially open.

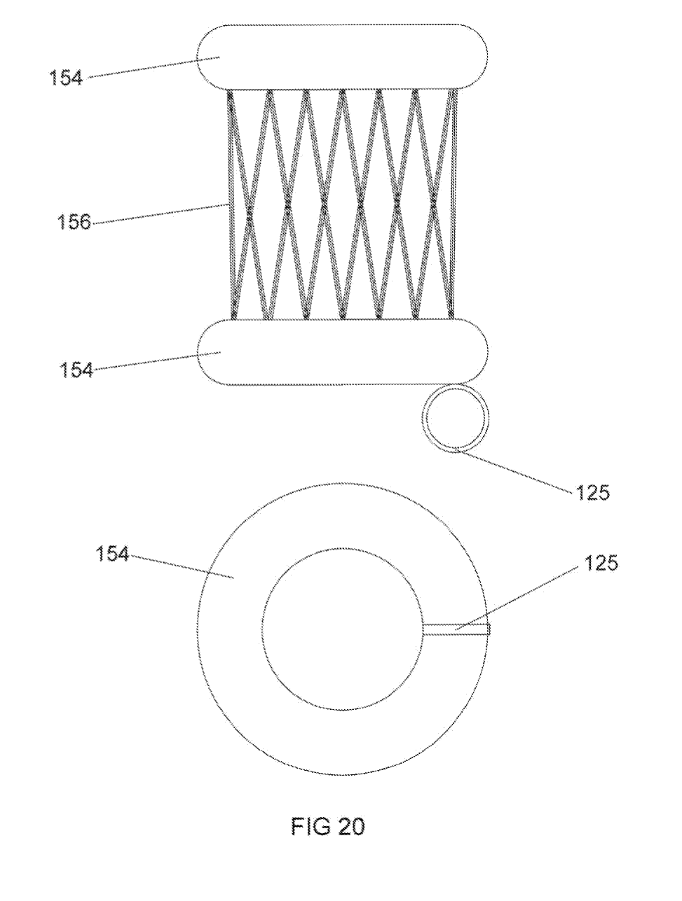

FIG. 20 shows an embodiment of the invention including two anchor rings 154 constructed of stranded Nitinol wire. The two rings are linked by longitudinal links 156. The longitudinal links 156 may overlap and form two different layers into a diamond-shaped mesh. The longitudinal links 156 may be constructed of solid Nitinol wire or stranded Nitinol wire or stranded other material such as stainless steel, Elgiloy, MP35N or L605. The longitudinal links may extend parallel to the longitudinal axis of the device or may run at an angle to the longitudinal axis. The longitudinal links 156 may be constructed integral with the anchor rings 154, from the same wire and wound at the same time or they may be made discretely and simply attached to the anchor rings 154. A removal loop 125 is attached to one of the anchor rings 154.

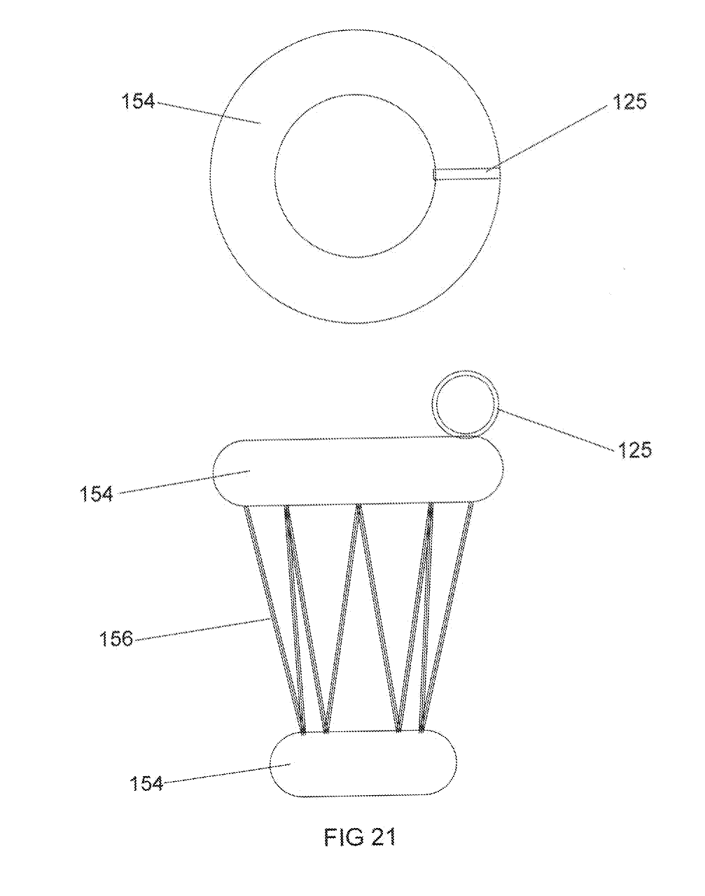

FIG. 21 shows an implant including a larger diameter anchor ring 154 and a smaller diameter anchor ring 154 constructed of stranded Nitinol wire. The anchor rings may be made of two different diameters to provide for anchoring at different lumen diameters. The anchor rings are linked by longitudinal links 156. The longitudinal links may be constructed of solid Nitinol wire or stranded Nitinol wire or stranded other material such as stainless steel, Elgiloy, MP35N or L605. The longitudinal links 156 may extend parallel to the longitudinal axis of the device or may run at an angle to the longitudinal axis. The longitudinal links 156 may be constructed integral with the anchor rings, from the same wire and wound at the same time or they may be made discretely and simply attached to the anchor rings. A removal loop is attached to one of the anchors rings 125.

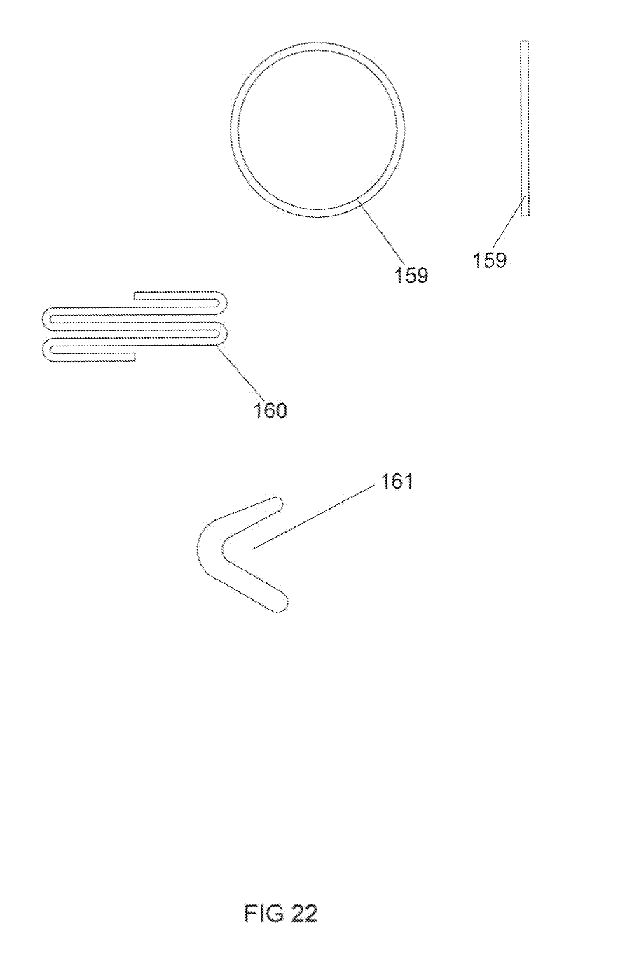

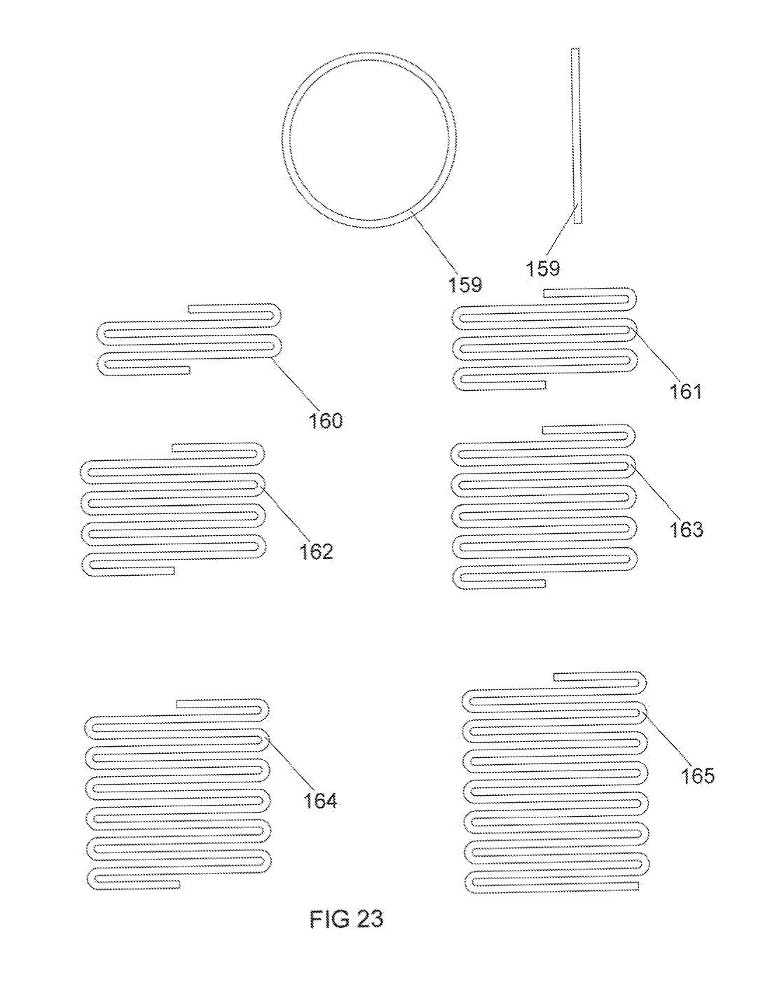

FIG. 22 shows an anchor ring 159 in the open state and in the collapsed state 160 ready to be loaded onto a delivery catheter. FIG. 23 shows an anchor ring 159 in the open state and 160, 161, 162, 163, 164 and 165 in alternative collapsed states ready to be loaded onto a delivery catheter.

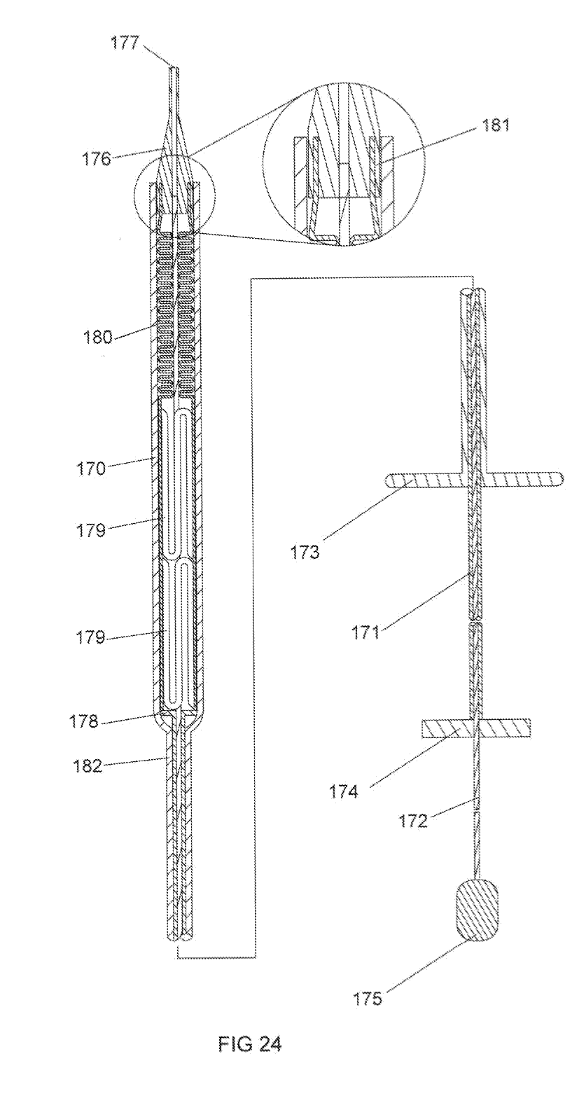

FIG. 24 is a cross-sectional view of a delivery catheter including the following three coaxial components: a distal outer sheath 170 which transitions down to a smaller diameter at the proximal outer sheath 182, a proximal pusher catheter 171, and a sleeve advancement pusher 172. The delivery catheter includes the following three handles: an outer sheath handle 173, a proximal pusher handle 174, and a sleeve advancement pusher handle 175. The implant pusher 178 serves as a mechanical stop or means to hold stationery or push out the anchor rings 179 or implant from the inside of the distal outer sheath 170. The distal tip 176 has two purposes. It provides for a flexible tip that will track over a guide wire. The guide wire may be inserted through the central lumen 177. The proximal shoulder of the tip 181 is rolled back over the end of the intestinal bypass sleeve 180 to constrain the intestinal bypass sleeve 180 to distal tip 176 and the sleeve advancement pusher 172 and to provide a mechanism of advancement of the intestinal bypass sleeve through the duodenum.

Two anchor rings 179 and the intestinal bypass sleeve 180 are compressed and loaded onto the delivery catheter. The intestinal bypass sleeve 180 can be folded and compressed into outer sheath 170. Alternatively the intestinal bypass sleeve 180 can simply extend in front of the outer sheath 170 and not be compressed or folded into the outer sheath 170. The intestinal bypass sleeve 180 can be attached to the distal tip 176 and sleeve delivery catheter 175 and may extend two feet (length the intestinal bypass sleeve 180) distal to the outer sheath 170.

The distal outer sheath 170 may be made from a plastic polymer such as Pebax (polyether block amide), hytrel (polyester elastomer), nylon 12, nylon 11, nylon 6, nylon 6,6, polyethylene, polyurethane or other suitable polymer. The distal outer sheath 170 may have an inner lining made from a polymer with a low coefficient of friction such as PTFE or ePTFE. The distal outer sheath 170 may also have a metal re-enforcement in the wall thickness to improve the kink resistance or burst properties of the outer sheath. The metal re-enforcement may be comprised of a braided wire mesh or a coil in the wall thickness. The metal used for the braid may be stainless steel, Nitinol, MP35n, L605, Elgiloy or other suitable material. The distal outer sheath 170 length may range from 1 inch long up to the full length of the catheter.

The proximal outer sheath 182 may be made from a plastic polymer such as Pebax (polyether block amide), Hytrel (polyester elastomer), nylon 12, nylon 11, nylon 6, nylon 6,6, polyethylene, polyurethane or other suitable polymer. The proximal outer sheath 182 may have an inner lining made from a polymer with a low coefficient of friction such as PTFE. The proximal outer sheath 182 may also have a metal re-enforcement in the wall thickness to improve the kink resistance or burst properties of the outer sheath. The metal re-enforcement may be comprised of a braided wire mesh or a coil in the wall thickness. The metal used for the braid may be stainless steel, Nitinol, MP35n, L605, Elgiloy or other suitable material.

The proximal pusher catheter 171 may be made from a plastic polymer such as Pebax (polyether block amide), Peek, Hytrel (polyester elastomer), nylon 12, nylon 11, nylon 6, nylon 6,6, polyethylene, polyurethane or other suitable polymer. The proximal pusher catheter 171 may have an inner lining made from a polymer with a low coefficient of friction such as PTFE. The proximal pusher catheter 171 may also have a metal re-enforcement in the wall thickness to improve the kink resistance or burst properties of the outer sheath. The metal re-enforcement may be comprised of a braided wire mesh or a coil in the wall thickness. The metal used for the braid may be stainless steel, Nitinol, MP35n, L605, Elgiloy or other suitable material

The sleeve advancement pusher 172 may be made from a plastic polymer such as Pebax (polyether block amide), Peek, Hytrel (polyester elastomer), nylon 12, nylon 11, nylon 6, nylon 6, 6, polyethylene, polyurethane or other suitable polymer. The sleeve advancement pusher 172 may have an inner lining made from a polymer with a low coefficient of friction such as PTFE. The sleeve advancement pusher 172 may also have a metal re-enforcement in the wall thickness to improve the kink resistance or burst properties of the outer sheath. The metal re-enforcement may be comprised of a braided wire mesh or a coil in the wall thickness. The metal used for the braid may be stainless steel, Nitinol, MP35n, L605, Elgiloy or other suitable material. The sleeve advanced pusher 172 may have a hollow core to allow passage over a guide wire or it may be solid without an opening. The sleeve advanced pusher 172 may also be constructed of a simple tightly wound metal wire coil construction or it may be wound from multiple wires such as Hollow Helical Strand tube made by Fort Wayne Metals of Fort Wayne, Ind. The sleeve advanced pusher 172. Sleeve advancement pusher handle 175 may also be comprised of a solid tube of Peek, Nitinol or stainless steel. The solid tube may have a series of slots or a patterned on a portion of the tube length to increase the flexibility of the component as required.

The distal tip 176 may be molded from Pebax, polyurethane, Hytrel or other suitable elastomer. The distal tip 176 had an outer flange 181 that is soft and may be rolled back and the intestinal bypass sleeve 180 may be inserted under it to secure the sleeve during transport to the distal duodenum.

Each of the handles 173, 174, 175 may be molded or machined from metal or plastic. The outer sheath handle 173 is attached to the proximal outer sheath 182. The outer sheath handle 173 is used to hold or retract the distal outer sheath 170 and the proximal outer sheath 182 during the advancement of the delivery catheter into the human anatomy, and while deploying the anchoring rings. The proximal pusher handle 174 is attached to the proximal pusher catheter 171. The outer sheath handle 173 is used to hold or push forward the proximal pusher catheter 171 and the implant pusher 178 during the advancement of the delivery catheter into the human anatomy, and while deploying of the anchoring rings.

The delivery catheter of FIG. 24 may be used with the following deployment sequence. The delivery catheter is preloaded with the anchor rings 179 and the intestinal bypass sleeve 180. The delivery catheter is advanced through the mouth, esophagus and stomach to the pylorus. The sleeve advancement pusher handle 175 is pushed distally while holding the rest of the catheter stationary. This pushes the sleeve advancement pusher handle 175, the distal tip 176 and the intestinal bypass sleeve 180 into the duodenum. The pusher handle 175 is further advanced until the intestinal bypass sleeve 180 reaches the ligament of treitz or the most distal location intended for the intestinal bypass sleeve 180. At this point all the slack in the sleeve 180 is taken up and outer sheath handle 173 is retracted towards the proximal pusher handle 174. The first anchor ring or distal anchor ring 179 is deployed or expanded distally to the pylorus. The entire delivery system is gently tension proximally to ensure that the distal expandable anchor is in contact with the pylorus. The outer sheath handle 173 is retracted further towards the proximal pusher handle 174 to unsheathe and expose the proximal anchor ring 179. The proximal anchor ring 179 self expands and opens up to its non-compressed (i.e., expanded) diameter once the sheath 170 is fully retracted. The delivery catheter can then be removed from the patient's stomach and esophagus.

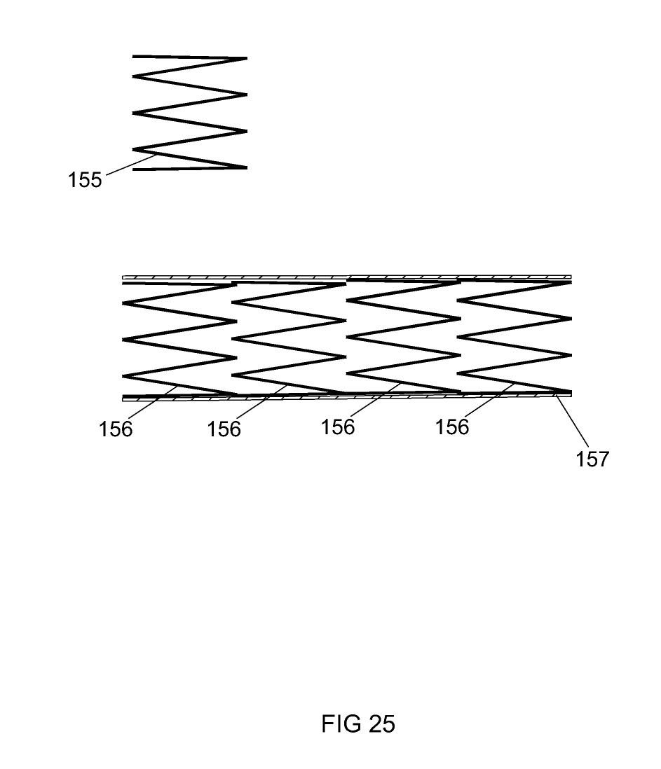

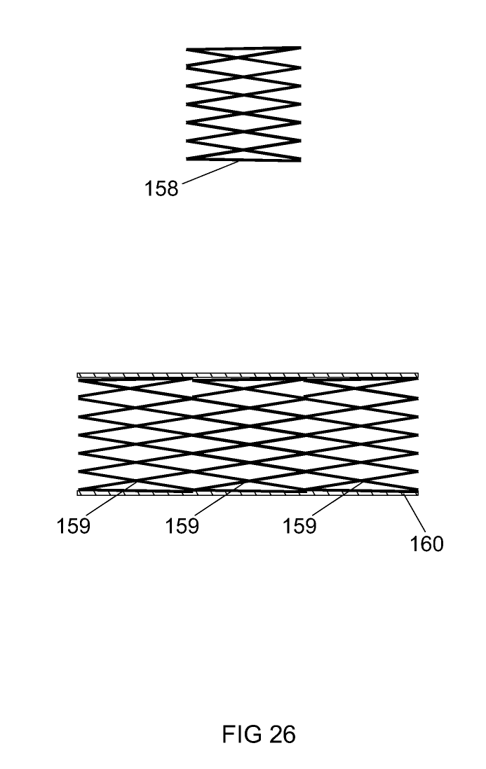

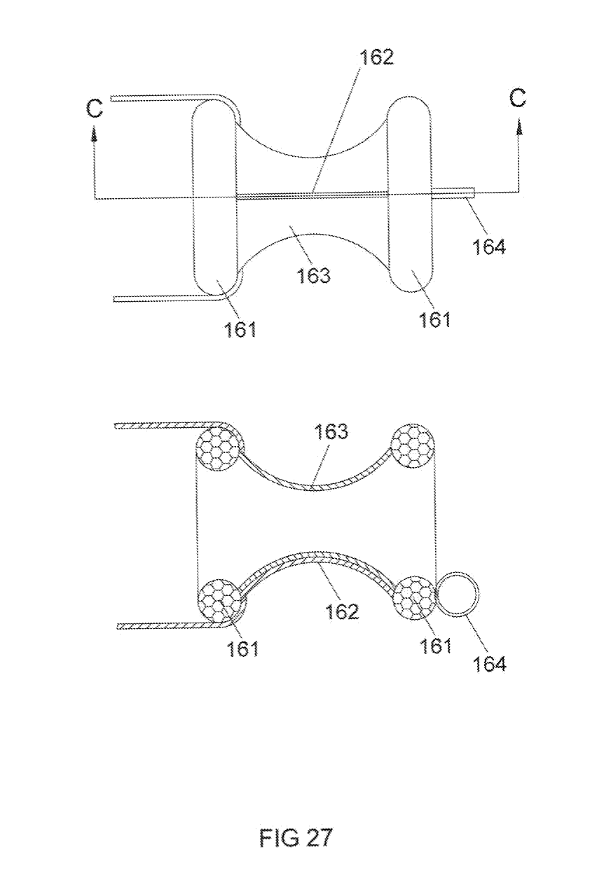

FIG. 25 shows a single stent or anchor 155 made from stranded wire. Multiple stents or anchors 156 that are linked by a sleeve 157. The sleeve 157 may be composed of silicone, polyurethane, ePTFE, Dacron, polyethylene or other suitable polymer. The sleeve may be loaded, impregnated or coated with a drug that may be eluted from the sleeve to provide for a therapeutic benefit. FIG. 26 shows a single stent or anchor 158 made of stranded wire, the anchors form a diamond lattice shape. Multiple stents or anchors 159 are linked by a sleeve 160. The sleeve 160 may be composed of silicone, polyurethane, ePTFE, Dacron, polyethylene or other suitable polymer. The sleeve may be loaded, impregnated or coated with a drug that may be eluted from the sleeve to provide a therapeutic benefit. FIG. 27 shows two anchor rings 161 linked by a flexible membrane 163. The two anchor rings 161 are also linked by a small diameter wire 162, near the removal loop 164. The small diameter wire 162 serves to provide a ridge tension link between the two anchor rings 161 and allows for easier collapse and removal of the device. FIG. 27 also shows a sectional view along the line C-C.

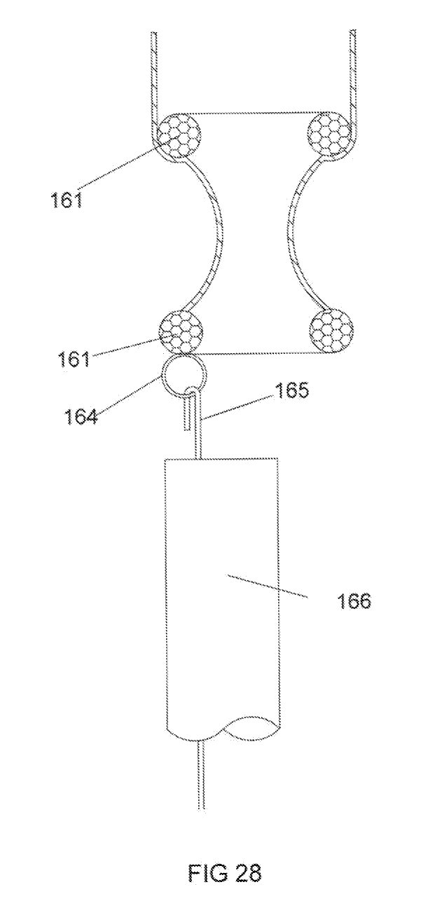

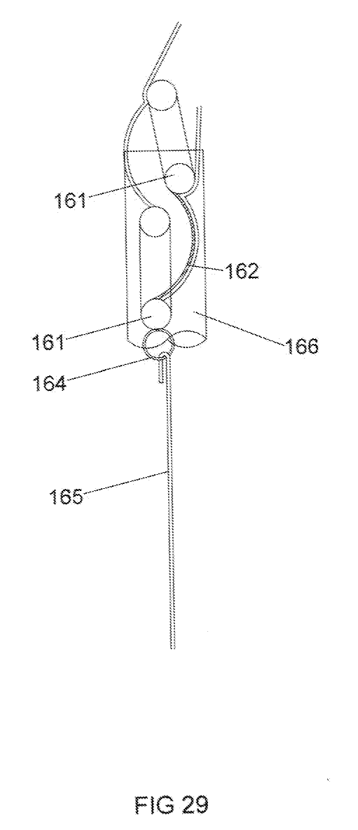

FIG. 28 shows a removal catheter/sheath 166 with a removal snare 165 attached to the removal loop 164 on the proximal anchor ring 161. FIG. 29 shows two anchor rings 161 collapsed and partially retrieved into the removal catheter 166 by pulling the removal loop 164 with removal snare 165.

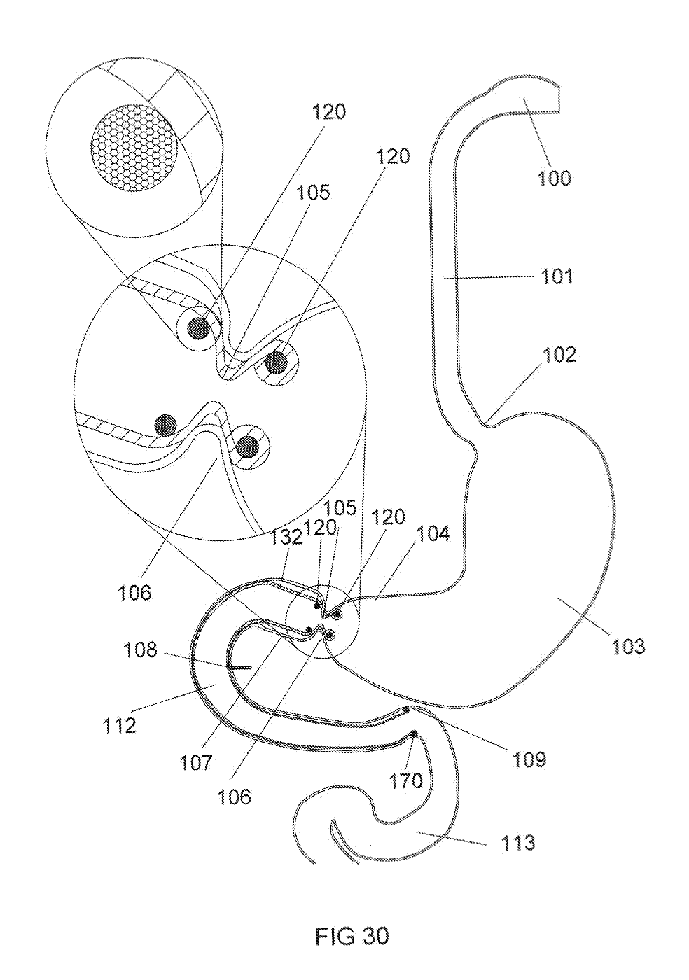

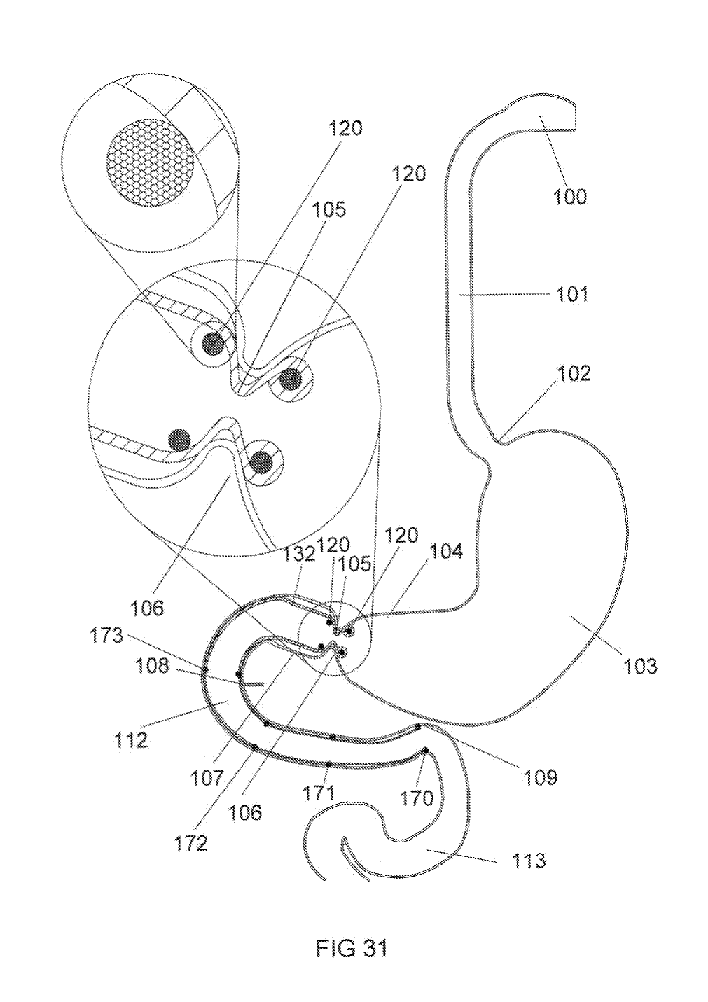

FIG. 30 is a sectional view of an embodiment of the invention implanted in a portion of a human digestive tract. As shown, an intestinal bypass sleeve 132 is implanted in the duodenum from the pylorus 106 to the ligament of treitz 109. The sleeve is held in place at the pylorus by two expandable anchors 120, one located on a first side of the pylorus and the second located on the opposite side of the pylorus. A third anchor ring 170 is attached to the intestinal bypass sleeve 132 near the ligament of treitz 109. FIG. 31 is a sectional view of an embodiment of the invention implanted in a portion of the digestive tract in a human body. As shown, an intestinal bypass sleeve 132 is implanted in the duodenum from the pylorus 106 to the ligament of treitz 109. The sleeve is held in place at the pylorus by two expandable anchors 120 one each side of the pylorus. A third anchor ring 170 is attached to the intestinal bypass sleeve 132 near the ligament of treitz 109. Additional anchor rings 171, 172, 173 are attached to the intestinal bypass sleeve 132 between the ligament of treitz 109 and the pylorus 106.

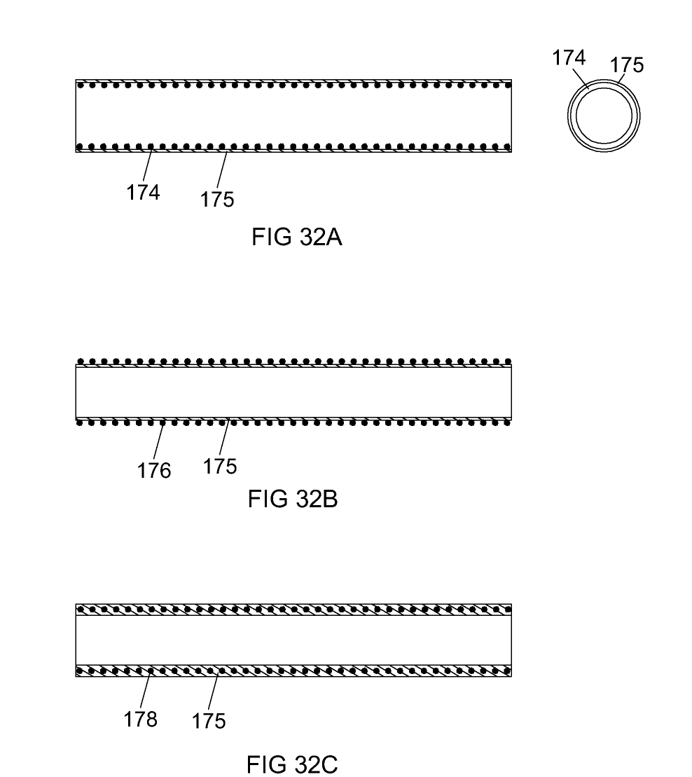

FIG. 32A shows a covered stent with the stent or anchor ring 174 made from stranded wire. The stent geometry (anchor ring) 174 may be in the form of simple rings, z-shaped elements, helically wound spiral coil or other suitable geometry. The anchor rings 174 are located on the inside of a polymer sleeve 175. The polymer sleeve 175 may be comprised of silicone, polyurethane, ePTFE, polyethylene, polypropylene, PTFE or other suitable polymer. FIG. 32B shows a covered stent with the stent or anchor ring 176 made from stranded wire. The stent geometry (anchor ring) 176 may be in the form of simple rings, z-shaped elements, helically wound spiral coil or other suitable geometry. The anchor rings 176 are located on the outside of a polymer sleeve 175. The polymer sleeve 175 may be comprised of silicone, polyurethane, ePTFE, polyethylene, polypropylene, PTFE or other suitable polymer. FIG. 32C shows a covered stent with the stent or anchor ring 178 made from stranded wire. The stent geometry (anchor ring) 178 may be in the form of simple rings, z-shaped elements, helically wound spiral coil or other suitable geometry. The anchor rings 178 are located within the wall thickness of the polymer sleeve 175. The polymer sleeve 175 may be comprised of silicone, polyurethane, ePTFE, polyethylene, polypropylene, PTFE or other suitable polymer.

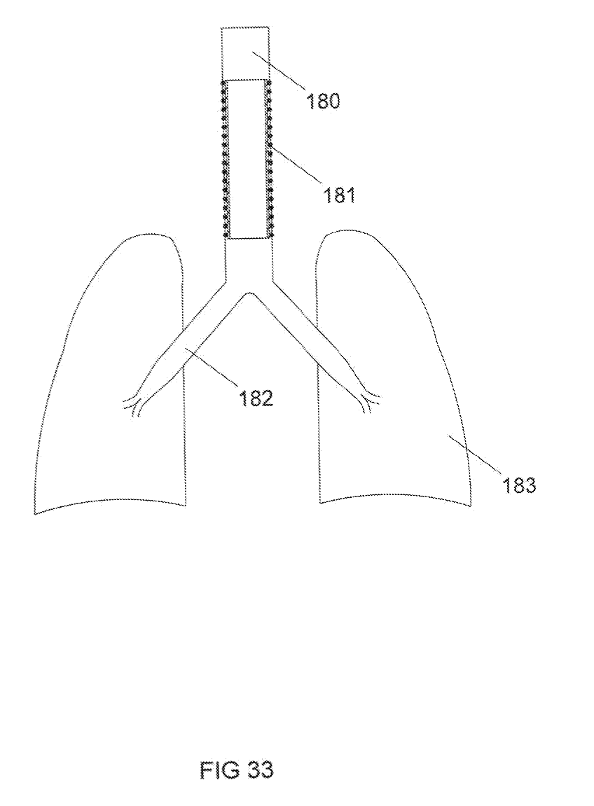

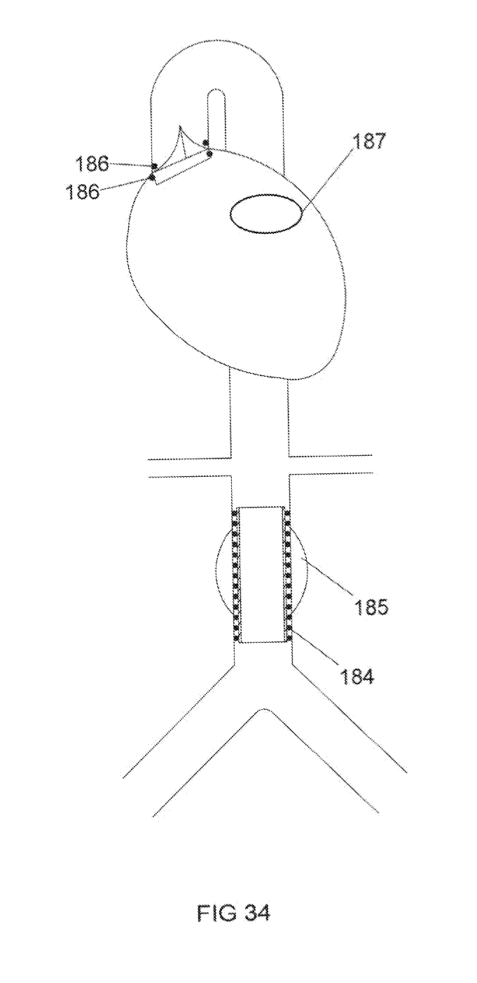

FIG. 33 shows a covered stent 181 as in FIGS. 32A, 32B and 32C implanted in the human body in the trachea 180. Other suitable implant locations are the bronchi 182 of the lung 183 or the bile duct, colon, duodenum or nasal duct. FIG. 34 shows a covered stent 184 as in FIGS. 32A, 32B and 32C implanted in the human body in an aortic aneurism 185. The stranded wire is also used as an anchor for a stent 186 for a percutaneously or trans-apically placed aortic or mitral heart valve, or for a mitral valve annuloplasty ring 187.

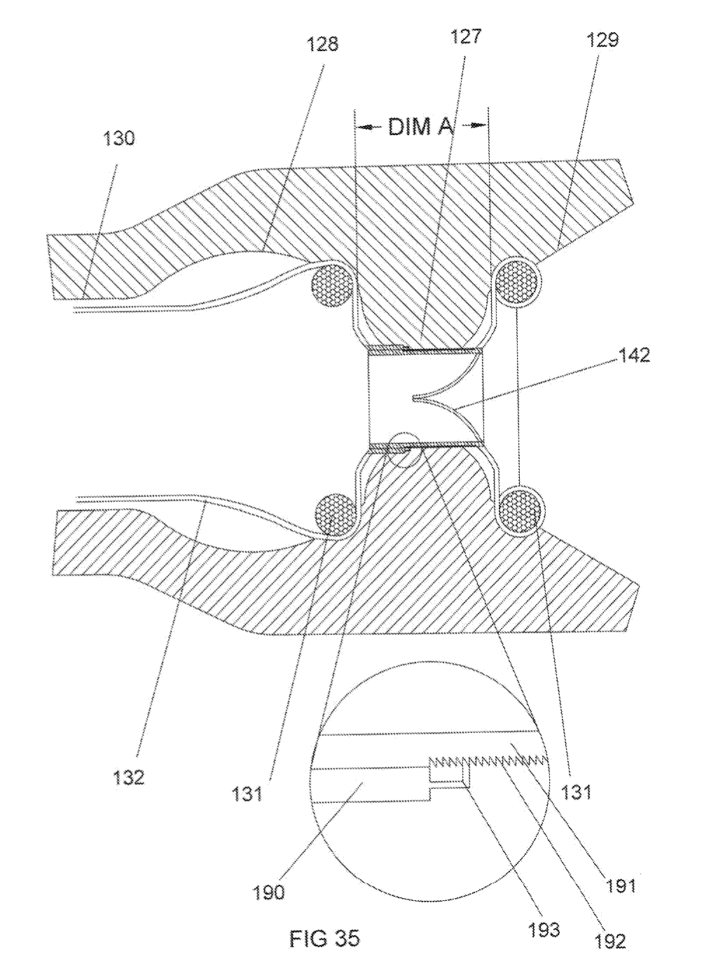

FIG. 35 is a cross-sectional view of an embodiment of the invention implanted into a pylorus 127, a duodenal bulb 128, and a duodenum 130. The rings are sized sufficiently large in diameter that there is a contact force between the ring diameter and the stomach pyloric antrum 129 and the duodenal bulb 128. The anchor rings 131 are sufficiently large in diameter that they are larger than the maximum opened diameter of the pylorus and therefore provide an anchoring mechanism to hold the intestinal bypass sleeve 132. The intestinal bypass sleeve can vary in length from a couple of inches in length up to several feet. In some embodiments, the sleeve bypasses the length of the duodenum up to the ligament of treitz. The intestinal bypass sleeve 132 can also be longer and bypass into the jejunum.

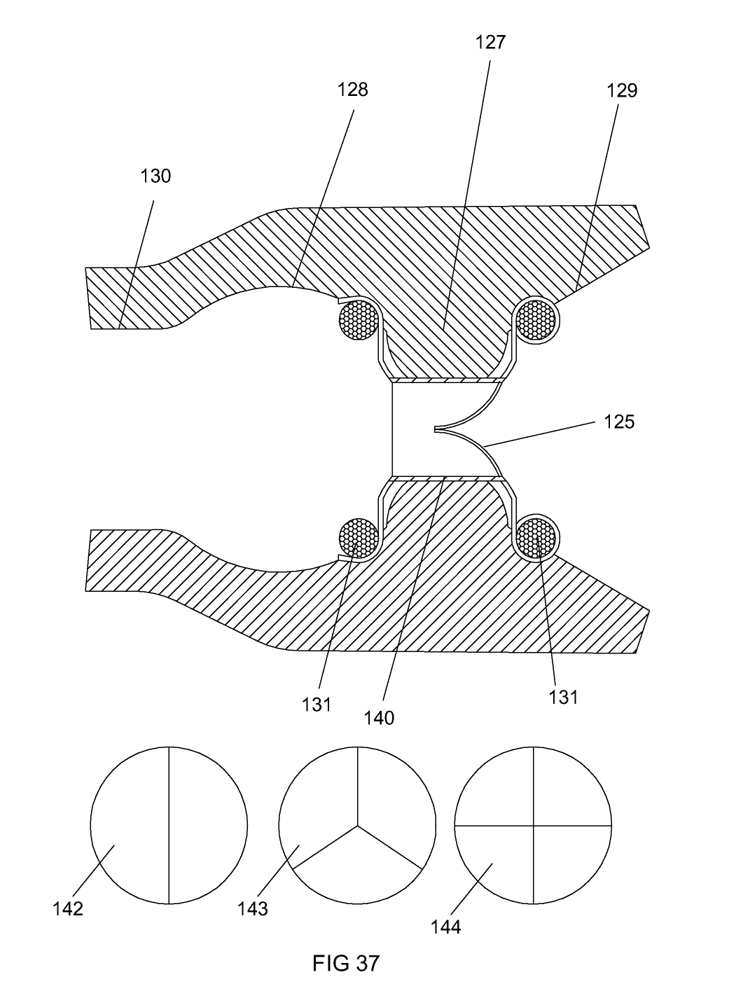

The pyloric portion of the device 190 and 191 disposed between the two anchor rings can be made from a rigid cylinder made from plastic material such as delrin, peek, high density polyethylene, polycarbonate or other suitable polymer. The pyloric portion 190, 191 may also be made from stainless steel, titanium or Nitinol. The fixed diameter of the pyloric portion pieces 190 and 191 of the device can be sized to provide for a full opening of the pylorus and not allow the pylorus to close normally. The length of the pyloric portion of the device (DIM A) can be adjusted by sliding the outer cylinder 190 over inner cylinder 191 by sliding on the ratcheting mechanism. This will change the spacing between the anchor rings 131 and will allow the device to be adjusted for ring spacing in-situ. It may be desirable to change the ring spacing to accommodate differences in the pylorus 127 dimensions from patient to patient. It may also be desirable to change the length of the pyloric portion, DIM A to allow the anchor ring 131 spacing ring to be adjusted to allow the ring to put a clamping force on to the pylorus in a longitudinal direction. The mechanism used for 192 and 193 could also be a screw thread arrangement such as a male thread on 192 and a female thread on 193. The inside diameter of the pyloric portion 190 and 191 could range from as small as 4 mm up to as large as 12 mm. The central lumen of device has a one way anti-reflux valve 142. The anti-reflux valve 142 allows for unobstructed flow in the direction of the stomach antrum 129 to the pylorus 127, but limits flow in the reverse direction. The anti-reflux valve 142 can be constructed of a duck bill design with two flexible leaflets, or may utilize other designs such as a tri-leaflet valve 143 or quad-leaflet valve 144 (see, e.g., FIG. 37). The anti-reflux valve may be constructed of silicone or polyurethane, polyethylene, ePTFE or any other suitable polymer.

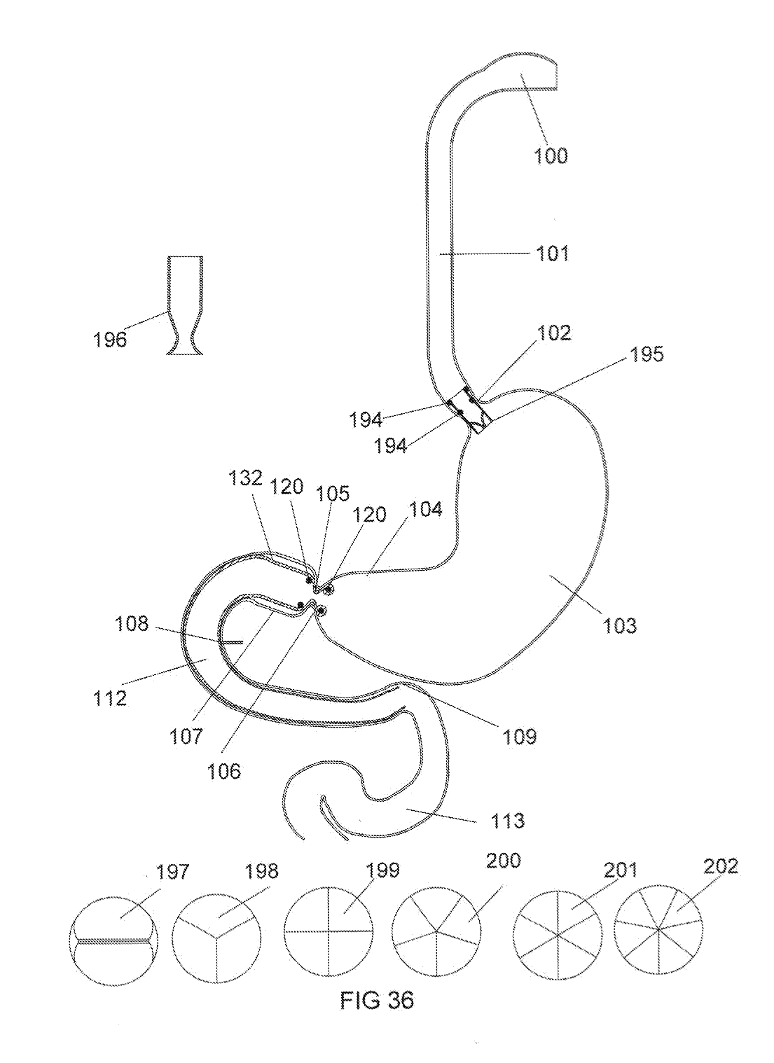

FIG. 36 is a sectional view of an embodiment of the invention implanted in a portion of the digestive tract in a human body. As shown, an intestinal bypass sleeve 132 is implanted in the duodenum from the pylorus 106 to the ligament of treitz 109. The sleeve is held in place at the pylorus by two expandable anchors 120 located on each side of the pylorus. An anti-reflux device 195 for GERD is implanted at the gastro-esophageal (GE) junction 102 and is held is place by two anchor rings 194. An alternative device that may be anchored at the GE junction 102 is a restrictive stoma 196. Items 197, 198, 199, 200, 201 and 202 are valve designs that may be used for the anti-reflux device 195.

FIG. 37 is a cross-sectional view of a device for the treatment of gastroparesis implanted into a pylorus 127, a duodenal bulb 128, and a pyloric antrum 129. The rings may be sized sufficiently large in diameter that there is a contact force between the ring diameter and the stomach pyloric antrum 129 and the duodenal bulb 128. The anchor rings 131 are sufficiently large in diameter that they are larger than the maximum opened diameter of the pylorus and therefore provide an anchoring mechanism to hold the gastroparesis device. The device may include an intestinal bypass sleeve that can vary in length from a couple of inches in length up to several feet.

In various embodiments, an intestinal bypass sleeve may not be required to treat gastroparesis. In other embodiments, for example to treat type 2 diabetes which is also prevalent in patients with gastroparesis, an intestinal bypass sleeve may be included. The pyloric portion 140 between the two anchor rings can be configured as a rigid (or semi-rigid) cylinder made from plastic material such as delrin, peek, high density polyethylene, polycarbonate or other suitable polymer. The pyloric portion may also be made from stainless steel, titanium or Nitinol. The fixed diameter of the pyloric portion 140 of the device can be sized to provide for a full opening of the pylorus and not allow the pylorus to close normally. In various embodiments, the pyloric portion 140 has sufficient stiffness or rigidity to maintain (or substantially maintain) the pylorus in its fully open configuration. In other embodiments, the pyloric portion 140 is configured to maintain (or substantially maintain) the pylorus in a partially open configuration.

In some embodiments, the diameter of the pyloric portion 140 is from as small as 3 mm in diameter up to as large as 18 mm in diameter. In other embodiments, the pyloric portion 140 could range from between about 8 mm and about 12 mm. The central lumen of device has a one way anti-reflux valve 142. The anti-reflux valve 142 allows for unobstructed flow in the direction of the stomach antrum 129 to the pylorus 127, but limits flow in the reverse direction. The anti-reflux valve 142 can be constructed of a duck bill design with two flexible leaflets, or may utilize other designs such as a tri-leaflet valve 143 or quad-leaflet valve 144. The anti-reflux valve may be constructed of silicone or polyurethane, polyethylene, ePTFE or other suitable polymer. The other device designs herein disclosed may also be suitable for use in a device for treatment of gastroparesis.

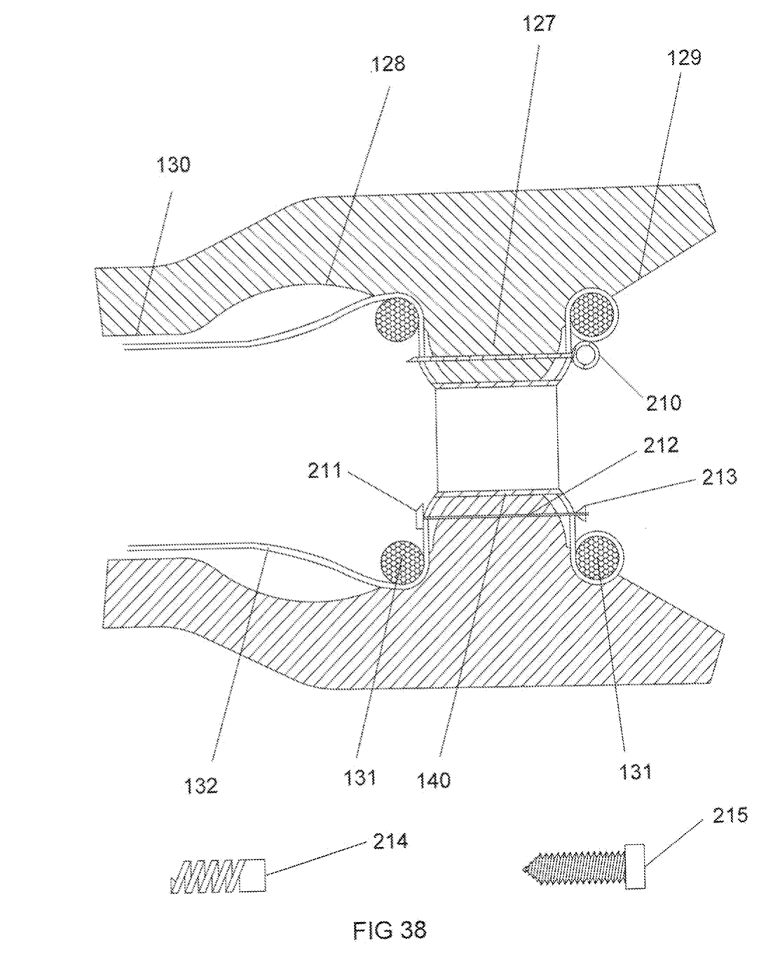

FIG. 38 is a cross-sectional view of an embodiment of the invention implanted into a pylorus 127, a duodenal bulb 128, and a duodenum 130. The rings are sized sufficiently large in diameter that there is a contact force between the ring diameter and the stomach pyloric antrum 129 and the duodenal bulb 128. The anchor rings 131 are sufficiently large in diameter that they are larger than the maximum opened diameter of the pylorus and therefore provide an anchoring mechanism to hold the intestinal bypass sleeve 132. The intestinal bypass sleeve can vary in length from 1-2 inches in length up to several feet. In some embodiments, the sleeve bypasses the length of the duodenum up to the ligament of treitz. The sleeve can also be longer and bypass into the jejunum. The pyloric portion of the device 140 located between the two anchor rings can be configured as a rigid cylinder made from plastic material such as delrin, peek, high density polyethylene, polycarbonate or other suitable polymer. The pyloric portion may also be made from stainless steel, titanium or Nitinol. The fixed diameter of the pyloric portion 140 of the device can be sized to provide for a full opening of the pylorus and not allow the pylorus to close normally. In some embodiments, the diameter of the pyloric portion 140 is from as small as 3 mm in diameter up to as large as 18 mm in diameter. In other embodiments, the pyloric portion 140 could range from between about 8 mm and about 12 mm. The central portion 140 may also be made compressible to allow smaller diameter loading profile when loaded onto a delivery catheter. A needle 210, suture (not shown), T-bar 211, hollow, helical anchor 214 or screw type anchor 215 is inserted into and or through the tissue of the pylorus to provide additional anchoring and securement of the intestinal bypass sleeve 132 anchoring device to the pylorus 127 anatomy. The T-bar 211 is anchored by a tensioning member 212 and cincher 213.

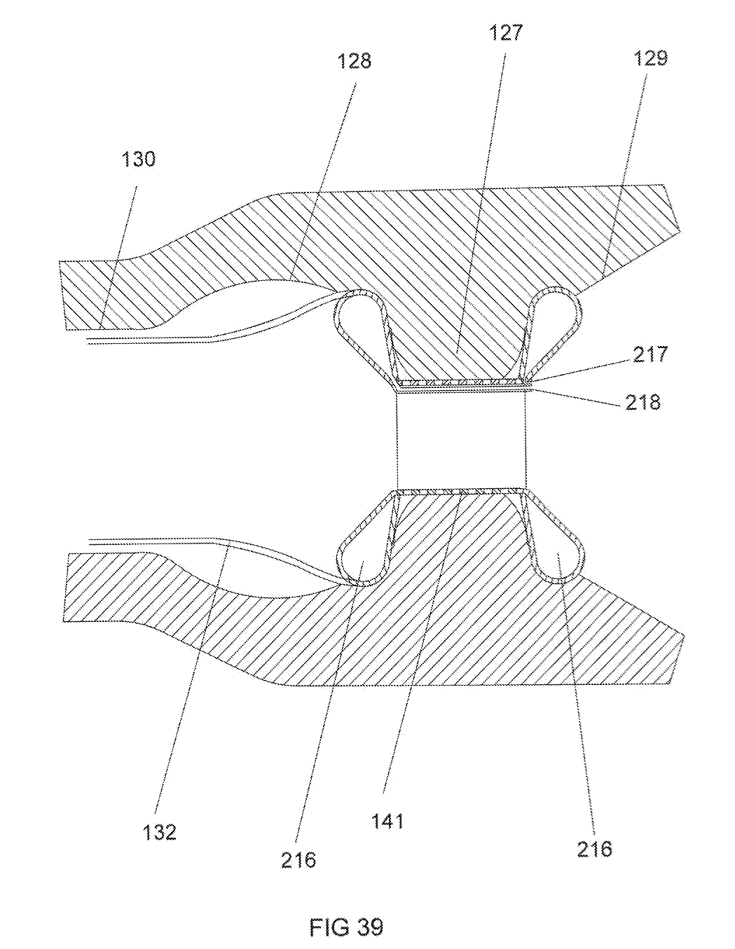

FIG. 39 is a cross-sectional view of an embodiment of the invention implanted into a pylorus 127, a duodenal bulb 128, and a duodenum 130. The anchoring device is comprised of two disk-shaped inflatable balloons 216 that are connected by a central cylinder 141. The two inflatable balloons 216 can be inflated through septum ports 217, 218. The balloons 216 may be filled with; a gas such as air or Carbon Dioxide (CO2), a liquid such as saline or polyethylene glycol, a liquid polymer such as silicone, polyurethane or epoxy, the liquid polymer cures into a gel or solid polymer after the balloon is filled. The central cylinder 141 is either fixed in diameter or its diameter can be compressed to reduce is diameter during delivery of the device to the implant location in the human body. The diameter of the central cylinder 141 will then elastically recover to its original diameter after it is released from the delivery catheter. The balloons 216 may be sized sufficiently large in diameter that there is a contact force between the balloon diameter and the stomach pyloric antrum 129 and the duodenal bulb 128. The balloons 216 are sufficiently large in diameter that they are larger than the maximum opened diameter of the pylorus 127 and therefore provide an anchoring mechanism to hold the intestinal bypass sleeve 132. The intestinal bypass sleeve can vary in length from 1-2 inches in length up to several feet. In some embodiments, the sleeve bypasses the length of the duodenum up to the ligament of treitz. The sleeve can also be longer and bypass into the jejunum. The pyloric portion 141 of the device between the two anchor balloons is constructed of an expandable diameter cylinder constructed of a stent-like construction. The cylinder has enough radial strength to resist compression by the pylorus when it closes, but provides for a central portion which can be compressed in diameter when it is loaded onto a delivery catheter so that the catheter diameter may be smaller. The central portion 141 may be self expanding or may require the use of a balloon to expand it. The central portion 141 may also be initially implanted at one diameter and then the diameter may be adjusted in size made larger or smaller if required because there is inadequate weight loss or a dumping syndrome in which the food moves too rapidly into the duodenum.

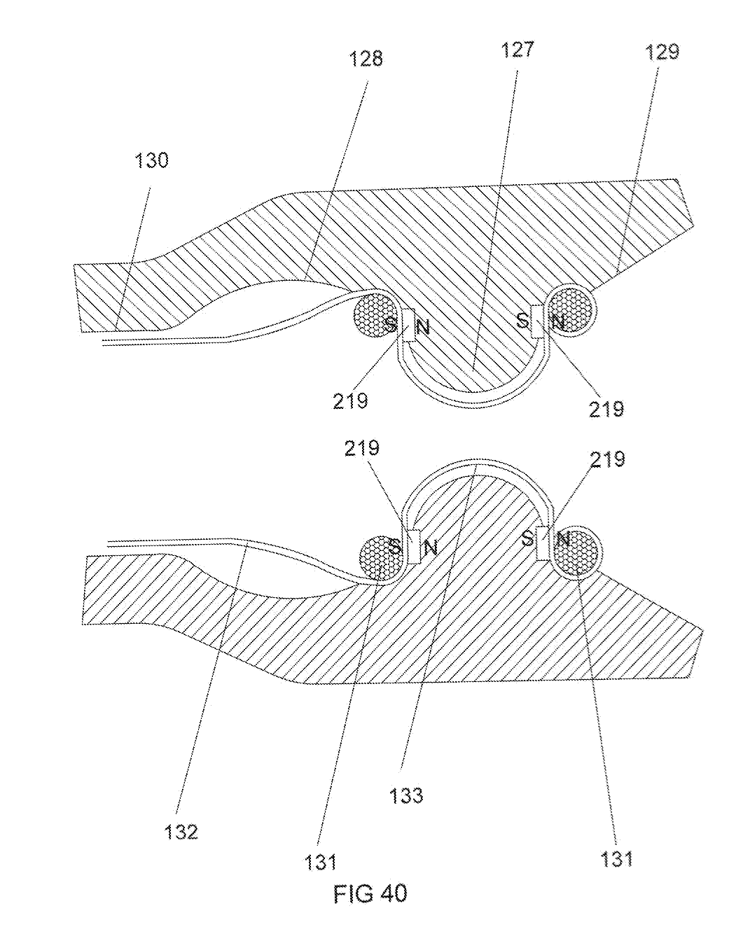

FIG. 40 is a sectional view of an embodiment of the invention implanted into a pylorus 127, a duodenal bulb 128, and a duodenum 130. The rings are sized sufficiently large in diameter to engage the wall of the stomach pyloric antrum 129 and the duodenal bulb 128. Magnets 219 are attached to anchor rings 131. The magnets 219 are attached to the anchor rings 131 so that the polarity (N, S) of the magnets is such that the magnets on the opposite sides of the pylorus are attracted to each other and exert a compressive force on the pylorus 127.

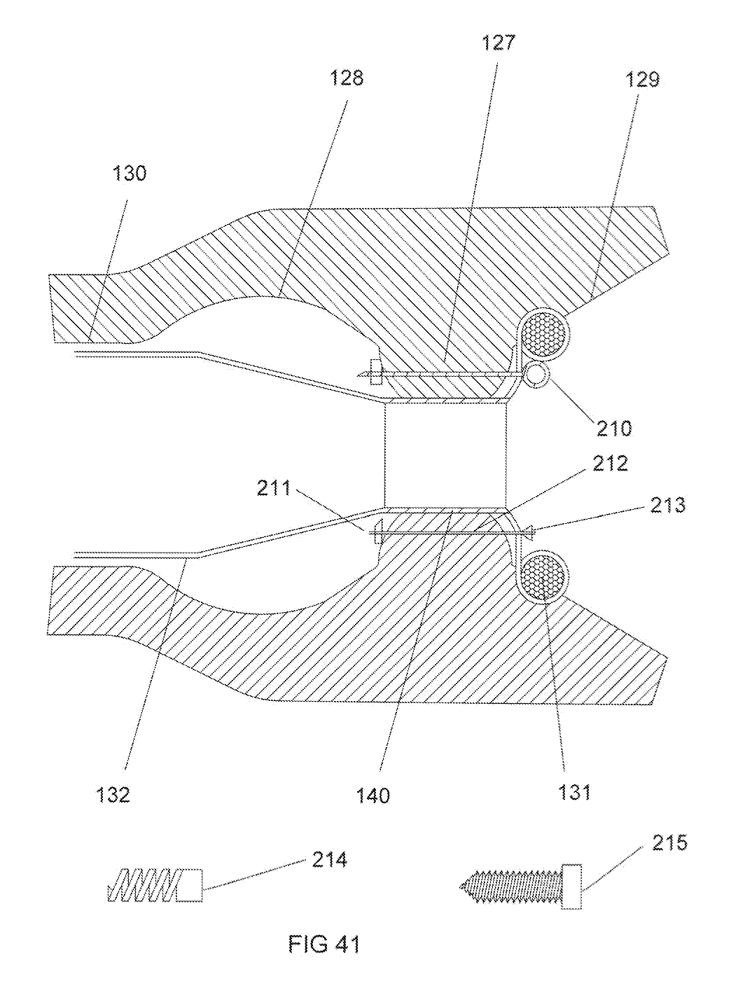

FIG. 41 is a sectional view of an embodiment of the invention implanted into a pylorus 127, a duodenal bulb 128, and a duodenum 130. An anchor ring 131 is sized sufficiently large in diameter to engage the wall of the stomach pyloric antrum 129. The central portion of the device is constructed of a rigid fixed-diameter cylinder 140 or can be a compressible cylinder. A needle 210, suture (not shown), T-bar 211, hollow helical anchor 214 or screw type anchor 215 is inserted into and/or through the tissue of the pylorus 127 to provide additional anchoring and securement of the intestinal bypass sleeve 132 anchoring device to pylorus anatomy or other suitable location. As shown, the T-bar 211 includes a pin 212 and a fastener 213. In other embodiments, the anchor ring 131 is located distal to the pylorus (within the duodenal bulb).

Various modifications and additions can be made to the exemplary embodiments discussed without departing from the scope of the present invention. For example, while the embodiments described above refer to particular features, the scope of this invention also includes embodiments having different combinations of features and embodiments that do not include all of the described features. Accordingly, the scope of the present invention is intended to embrace all such alternatives, modifications, and variations as fall within the scope of the claims, together with all equivalents thereof.

We claim:

1. A gastrointestinal device for implanting within a pylorus, a duodenal bulb, and a duodenum of a patient's gastrointestinal tract, the implant comprising:
a first expandable anchoring ring formed from stranded wire, the first expandable anchoring ring having a collapsed configuration and an expanded configuration, wherein in the expanded configuration the first expandable anchoring ring has a diameter larger than a maximum opening diameter of the pylorus;
a second expandable anchoring ring formed from stranded wire, the second expandable anchoring ring having a collapsed configuration and an expanded configuration, wherein in the expanded configuration the second expandable anchoring ring has a diameter larger than a maximum opening diameter of the pylorus; and
an intestinal bypass sleeve coupled to the first expandable anchoring ring and the second expandable anchoring ring, so as to allow a distance between the anchoring rings to exceed a width of the pylorus.

2. The device of claim 1 wherein the stranded wire ring is made from Nitinol.

3. The device of claim 2 wherein at least one of the first and second expandable anchoring rings is wound from 1×7 Nitinol stranded cable and further wherein the expandable anchoring ring has an outer diameter of between about 10 and about 60 mm.

4. The device of claim 2 wherein at least one of the first and second expandable anchoring rings is wound from a plurality of Nitinol wires, each of the plurality of wires having a diameter of between about 0.001 and about 0.005 inches, further wherein the expandable anchoring ring has an outer diameter of between about 10 and about 60 mm.

5. The device of claim 1 wherein the highly stranded wire is made from stainless steel.

6. The device of claim 1 further comprising a pyloric portion extending between and coupling the first anchoring ring and the second anchoring ring.

7. The device of claim 6 wherein the pyloric portion is comprised of a rigid material, such that it provides for a fixed pyloric orifice diameter.

8. The device of claim 7 wherein the pyloric portion includes a valve for preventing reflux of the duodenal contents back into the stomach.

9. The device of claim 6 wherein the pyloric portion is made from a plurality of longitudinal links extending between the first and second anchoring rings.

10. The device of claim 1 further including a supplemental securement mechanism, adapted for securing the pyloric portion to the pylorus, selected from the group consisting of: a suture, a t-bar mechanism, and a screw.

11. The device of claim 1 wherein at least one of the first and second expandable anchoring rings has an expanded outer diameter less than an inner diameter of the duodenal bulb, such that the expandable anchoring ring does not impose a force against the duodenal bulb.

12. An anchoring device for implanting within a pylorus for treating gastroparesis, the device comprising:
a first expandable anchoring ring having a collapsed configuration and an expanded configuration, wherein in the expanded configuration the first expandable anchoring ring has a diameter larger than a maximum opening diameter of the pylorus;
a second expandable anchoring ring having a collapsed configuration and an expanded configuration, wherein in the expanded configuration the second expandable anchoring ring has a diameter larger than a maximum opening diameter of the pylorus; and
the two anchoring rings are connected by a pyloric portion, the pyloric portion having a length greater than a width of the pylorus, such that the first expandable anchoring ring may be disposed on a first side of the pylorus and a second expandable anchoring ring may be disposed on a second side of the pylorus;
wherein the pyloric portion is configured to resist closure of the pylorus.

13. The device of claim 12 further including a valve coupled to the pyloric portion and adapted to allow flow through the pyloric portion in a first direction and to resist flow through the pyloric portion in a second direction

14. The device of claim 12 wherein at least one of the first and second expandable anchoring rings is formed from a highly stranded Nitinol wire.

15. The device of claim 12 wherein at least one of the first and second expandable anchoring rings is wound from 1×7 Nitinol stranded cable and further wherein the expandable anchoring ring has an outer diameter of between about 10 and about 60 mm.

16. The device of claim 13 wherein at least one of the first and second expandable anchoring rings is wound from a plurality of Nitinol wires, each of the plurality of wires having a diameter of between about 0.001 and about 0.005 inches, further wherein the expandable anchoring ring has an outer diameter of between about 10 and about 60 mm.

17. The device of claim 12 wherein at least one of the first and second expandable anchoring rings is formed from a disk-shaped inflatable balloon.

18. The device of claim 13 further including a supplemental securement mechanism, adapted for securing the pyloric portion to the pylorus, selected from the group consisting of: a suture, a t-bar mechanism, and a screw.

19. A method for treating gastroparesis comprising:
compressing a first expandable anchoring ring in a delivery tool, the first expandable anchoring ring having a collapsed configuration and an expanded configuration, wherein in the expanded configuration the first expandable anchoring ring has a diameter larger than a maximum opening diameter of the pylorus;
compressing a second expandable anchoring ring in the delivery tool, the second expandable anchoring ring having a collapsed configuration and an expanded configuration, wherein in the expanded configuration the second expandable anchoring ring has a diameter larger than a maximum opening diameter of the pylorus;
advancing the delivery tool to a pylorus of a patient;
deploying the second expandable anchoring ring on a first side of the pylorus;
deploying the first expandable anchoring ring on a second side of the pylorus; and
coupling a valve coupled to a pyloric portion extending between the first and second expandable anchoring rings, the valve adapted to allow flow through the pyloric portion in a first direction and to resist flow through the pyloric portion in a second direction.

20. The method of claim 19 wherein the delivery tool includes a distal outer sheath, a proximal outer sheath, a proximal pusher catheter, and a sleeve advancement pusher catheter and further wherein the delivery catheter includes an outer sheath handle, a proximal pusher handle, and a sleeve advancement pusher handle.
